SEQUENCE LISTING (19 sequences)

SEQ ID NO: 1 - protein, 18 aa, synthetic construct
MALPVTALLLPLALLLHA

SEQ ID NO: 2 - protein, 20 aa, synthetic construct
METDTLLLWVLLLWVPGSTG

SEQ ID NO: 3 - protein, 20 aa, synthetic construct
MGWSCIILFLVATATGVHSD

SEQ ID NO: 4 - protein, 238 aa, synthetic construct
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIK

SEQ ID NO: 5 - protein, 250 aa, synthetic construct
MDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 6 - protein, 242 aa, synthetic construct
DIELTQSPASLAVSLGQRAIISCKASQSVSFAGTSLMHWYHQKPGQQPKLLIYRASNLEAGVPTRFSGSGSKTDFTLNIHPVEEEDAATYYCQQSREYPYTFGGGTKLEIKGSTSGSGKSSEGKGQVQLQQSGAELVKPGASVKISCKASGYSFTGYFMNWVKQSHGKSLEWIGRIHPYDGDTFYNQNFKDKATLTVDKSSNTAHMELLSLTSEDFAVYYCTRYDGSRAMDYWGQGTTVTVS

SEQ ID NO: 7 - protein, 525 aa, synthetic construct
MNSGLQLVFFVLTLKGIQGEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKGEAAAKEAAAKEAAAKDIELTQSPASLAVSLGQRAIISCKASQSVSFAGTSLMHWYHQKPGQQPKLLIYRASNLEAGVPTRFSGSGSKTDFTLNIHPVEEEDAATYYCQQSREYPYTFGGGTKLEIKGSTSGSGKSSEGKGQVQLQQSGAELVKPGASVKISCKASGYSFTGYFMNWVKQSHGKSLEWIGRIHPYDGDTFYNQNFKDKATLTVDKSSNTAHMELLSLTSEDFAVYYCTRYDGSRAMDYWGQGTTVTVSSGSGHHHHHH

SEQ ID NO: 8 - protein, 16 aa, synthetic construct
GEAAAKEAAAKEAAAK

SEQ ID NO: 9 - protein, 16 aa, synthetic construct
GEAAAKEAAAKEAAAK

SEQ ID NO: 10 - protein, 537 aa, synthetic construct
MNSGLQLVFFVLTLKGIQGMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGEAAAKEAAAKEAAAKDIELTQSPASLAVSLGQRAIISCKASQSVSFAGTSLMHWYHQKPGQQPKLLIYRASNLEAGVPTRFSGSGSKTDFTLNIHPVEEEDAATYYCQQSREYPYTFGGGTKLEIKGSTSGSGKSSEGKGQVQLQQSGAELVKPGASVKISCKASGYSFTGYFMNWVKQSHGKSLEWIGRIHPYDGDTFYNQNFKDKATLTVDKSSNTAHMELLSLTSEDFAVYYCTRYDGSRAMDYWGQGTTVTVSSGSGHHHHHH

SEQ ID NO: 11 - DNA, 53 bp, synthetic construct
  source 1..53: mol_type = other DNA
tgtcgtgaaaactaccccgcggccgccaccatggctcagcggatgacaacaca

SEQ ID NO: 12 - DNA, 42 bp, synthetic construct
  source 1..42: mol_type = other DNA
ttcgtggctccggagccactgctgagcagccacagcagcatt

SEQ ID NO: 13 - protein, 14 aa, synthetic construct
GSTSGSGKSSEGKG

SEQ ID NO: 14 - protein, 18 aa, synthetic construct
MNSGLQLVFFVLTLKGIQ

SEQ ID NO: 15 - protein, 10 aa, synthetic construct
SGSGHHHHHH

SEQ ID NO: 16 - protein, 8 aa, synthetic construct
RAKRSGSG

SEQ ID NO: 17 - protein, 4 aa, synthetic construct
SGSG

SEQ ID NO: 18 - protein, 8 aa, synthetic construct
GGGGSAAA

SEQ ID NO: 19 - protein, 20 aa, synthetic construct
GGGGSGGGGSGGGGSGGGGS

(19) United States
(12) Patent Application Publication
(10) Pub. No.: US 2024/0408202 A1
(43) Pub. Date: Dec. 12, 2024

(54) FOLATE RECEPTOR ALPHA -TARGETING BI-SPECIFIC T CELL ENGAGERS (BiTEs) AND USES THEREOF

(71) Applicant: Health Research, Inc., Buffalo, NY (US)

(72) Inventors: Kunle ODUNSI, Oak Park, IL (US); Robert MCGRAY, Buffalo, NY (US); Takemasa TSUJI, Munster, IN (US)

(21) Appl. No.: 18/839,601

(22) PCT Filed: Feb. 27, 2023
(86) PCT No.: PCT/US2023/063359

§ 371 Date: Aug. 19, 2024

Related U.S. Application Data
(60) Provisional application No. 63/314,448, filed on Feb. 27, 2022.

(51) Int. Cl. A61K 39/00; A61P 35/00; C07K 16/28; C12N 5/0783; C12N 15/86

(52) U.S. Cl. CPC A61K 39/4611; A61K 39/4633; A61K 39/464402; A61K 39/464411; A61P 35/00; C07K 16/28; C07K 16/2809; C12N 5/0636; C12N 15/86; A61K 2239/59; C07K 2317/31; C07K 2317/622; C12N 2510/00; C12N 2740/10043

(57) ABSTRACT

Provided are modified cells and methods for their use in treating cancer. The cells are modified to express and secrete a Bi-specific T cell engager (BiTE) that includes a segment that specifically binds to human Folate Receptor alpha (FRα) and a segment that that specifically binds to human CD3, such as CD3e. The modified cells can be T cells. Methods for producing the modified cells are also provided.

### CROSS REFERENCE TO RELATED APPLICATION

This application claims priority to U.S. provisional patent application No. 63/314,448, filed Feb. 27, 2022, the entire disclosure of which is incorporated herein by reference.

### SEQUENCE LISTING

The instant application contains a sequence listing which has been submitted in .xml format and is hereby incorporated by reference in its entirety. Said .xml file is named “003551_01077_ST26.xml”, was created on Feb. 24, 2023, and is 19,383 bytes in size.

### RELATED INFORMATION

Cancer immunotherapies, including adoptive T cell transfer (ACT), have demonstrated impressive clinical activity, however their benefit in ovarian cancer (OC) and other cancers has generally been limited. While effective in hematological cancers, ACT has shown modest clinical impact when treating solid tumors¹, with the notable exception of autologous tumor-infiltrating T cells (TILs) where clinical responses have been achieved in melanoma and less frequently in other cancers²⁻⁴. In OC, the limited impact of ACT likely arises from the immunosuppressive tumor microenvironment (TME)⁵⁻⁷. Additionally, although TIL abundance correlates with improved survival in OC 8, recent evidence suggests most CD8+ TILs in OC patient tumors do not recognize cancer cells 9, instead comprised predominantly of bystander TILs¹⁰. Bystander TILs do not upregulate inhibitory receptors and persist as functional effector T cells¹¹. Therefore, ACT-based approaches that effectively engage and mechanistically redirect bystander TILs for antitumor immunity are likely to overcome local immune suppression and enhance tumor attack.

Bispecific T cell engagers (BiTEs) can redirect T cells for antigen-specific targeting¹² and are currently in development for OC¹³⁻¹⁵ However, conventional BiTEs have an intrinsically short circulating half-life¹⁶, necessitating repeated or continuous infusion to achieve therapeutic BiTE exposure, in addition to a prerequisite for adequate intratumoral T cell availability to elicit responses¹⁷. To overcome these limitations, generating BiTE-secreting T cells (BiTE-T cells) has emerged as a promising modality¹⁸⁻²¹, where unlike conventional CAR- or TCR-engineering strategies, BiTE-T cells secrete BiTEs to redirect both BiTE-T cells and host T cells, thereby magnifying therapeutic responses.

When assessing target antigens for ACT with broad expression in OC that can be targeted without severe risk of on target/off tumor toxicity, folate receptor alpha (FRα) has emerged as an optimal target. FRα is expressed by most epithelial OC cells²²23 with restricted normal tissue expression and has been associated with OC relapse and chemotherapy resistance²⁴. Further, targeting FRα using multiple therapeutic approaches have been or are being tested clinically^{23 25-27}, collectively demonstrating encouraging clinical responses and a generally favorable safety profile as highlighted by the recent FDA accelerated approval of the FRα-targeted antibody drug conjugate (ADC) mirvetuximab soravtansine (MIRV)²⁸. However, durable and/or broadly curative therapies targeting FRα in OC have not been identified, suggesting innovative strategies that integrate multiple approaches to enhance FRα targeting are needed to improve outcome. The present disclosure is related to this need.

### BRIEF SUMMARY

The present disclosure relates to modifying T cell to secrete BiTEs so that the modified T cells can be used as an adoptive cell therapy. In embodiments, one binding portion of the described BiTEs targets Folate Receptor alpha (FRα). The disclosure demonstrates that BiTE-secreting T cells (BiTE-T cells) can overcome challenges of durable BiTE delivery, which has previously been common to soluble BiTE formats. The present disclosure also overcomes the requirement for repeated (such as daily infusions) BiTEs that are common in preclinical tumor models. In representative and non-limiting demonstrations, the disclosure demonstrates that BiTE-T cells can be efficiently generated using retroviral transduction. The disclosure demonstrates that BiTE-T cells redirect BiTE T cells and non-transduced bystander T cells, leading to activation and robust target cell killing in an antigen-dependent manner. BiTE-T cells modified to contain an FRα targeted BiTE (FR-B; FR-B T cells) have therapeutic efficacy in multiple pre-clinical tumor models, with prolonged efficacy dependent on endogenous T cells. The disclosure demonstrates that, when delivered via loco-regional injection, FR-B-T cells can mediate potent anti-tumor immunity in the absence of systemic inflammation. Further, BiTE-T cell persistence following tumor antigen encounter can be improved through preconditioning of the T cells, illustrated using certain cytokines, such as Interleukin 15 (IL-15). This approach improves BiTE T cell persistence and therapeutic efficacy compared to preconditioning with IL-2+IL-7. The disclosure demonstrates that BiTE T cells can be delivered as a tuneable cell therapy using multi-dosing to enhance therapeutic efficacy.

### BRIEF DESCRIPTION OF THE DRAWINGS

Where reference to a figure number includes a letter the letter means a panel on the numbered figure.

FIG. 1: FR-Bh T cells target FRα+ tumor cells and initiate antitumor immune responses against OC patient specimens. A) Representative FACs plots demonstrating efficient production of FR-Bh T cells via retroviral transduction B) % SKOV-6 target cell lysis (left) and IFN-γ production (right) following 24 hr co-culture with FR-Bh or CONT-ENG T cells at specified E:T ratios (n=3/condition) C) % FRα+ cancer cells across tested OC patient specimens (n=10) D-F) FRα+ and FRα−tumor cell number and corresponding IFN-γ production (●(black)) from 48 hr OC patient co-cultures following the addition of CONT-ENG or FR-Bh T cells. Baseline tumor cell number and IFN-γ (co-cultures containing endogenous TALs only) shown for comparison. Individual patients and FRα status as shown. G) Heatmap showing relative changes and average Log₁₀ fold change for inflammatory factors following addition of CONT-ENG or FR-Bh T cells to patient co-cultures (n=4 patients). Data presented as mean±SEM. Data in B) is from one representative experiment. Data in B) Two-Way ANOVA & G) Paired t test (two-tailed), * p<0.05, *** p<0.001.

FIG. 2: FR-Bh T cells and endogenous OC patient T cells are activated by BiTEs when directed against FRα+OC patient samples. A) Representative FACs plots showing surface expression of activation markers (CD25, CD69, CD137, PD-1) for CD8+ CONT-ENG or FR-Bh T cells following 48 hr co-culture with a FRα+OC patient sample B) Graphical representation of data in A) across all FRα+ patients (upper, n=7) and Frα^{lo} patients (lower, n=3). C) Data as in A) for endogenous CD8+ tumor associated lymphocytes (TALs) D) Graphical representation of data in C) across all FRα+ patients (n=7). Baseline activation (endogenous T cells only) was used for comparison. For Data in B) and D), connected data points and unique colors correspond to individual OC patients. Data in B) Unpaired & D) Paired t test (two-tailed), * p<0.05, ** p<0.01, *** p<0.001.

FIG. 3: Therapeutic Delivery of murine FR-B T cells improves tumor control and survival in OC tumor-bearing mice A) Representative FACs plots demonstrating efficient production of FR-B CD8+ T cells via retroviral transduction. Untransduced (UTD) CD8+ T cells shown as a control B) IE9-mp1-hFRa target cell lysis (left, n=4 ROI/condition) and IFN-γ production (right, n=3 replicates/condition) following 24 hr co-culture with FR-B or Unarmed Control T cells at specified E:T ratios. Parental IE9-mp1 cells (hFRα−) were used as a target antigen negative control. C) Activation of GFP+ Transduced T cells (FR-B or Luc/GFP) and GFP-untransduced (UTD) bystander T cells based on CD69 surface staining 24 hrs following co-culture with IE9-mp1-hFRa cells. D) Experimental Design (left) and survival (right) of IE9-mp1-hFRa tumor bearing mice treated locoregionally with FR-B T cells or Unarmed Control T cells by IP injection (n=10-11/group). Data presented as mean±SEM. Data in A-C is from one representative experiment. Data in D) compiled from 2 independent experiments. Data in B) Two-Way ANOVA & D) Log-rank Test, * p<0.05, ** p<0.01, *** p<0.001.

FIG. 4: Preconditioning FR-B T cells with IL-2 & IL-15 produces stem-like FR-B T cells with reduced effector function but enhanced persistence and antitumor activity when adoptively transferred: Representative FACs plots showing A) similar transduction efficiencies between FR-B 2/7 and FR-B 2/15 CD8+ T cells using retroviral transduction with untransduced (UTD) CD8+ T cells shown as a control and B) Increased frequency of TCF-1^{hi} FR-B CD8+ and CD4+ cells in 2/15 conditioned T cells C) Mitochondrial respiration of FR-B 2/7 and FR-B 2/15 T cells measured by Mitochondrial Stress Test using the Seahorse XFe96 Analyzer (n=8/group) D) IFN-γ production (left, n=3 replicates/condition) and IE9-mp1-hFRa target cell lysis (right, n=4 ROI/condition) following 48 hr co-culture with FR-B 2/7, FR-B 2/15, or Unarmed Control T cells. E) Enumeration of FR-B T cells following serial co-culture with IE9-mp1-hFRa target cells at a fixed 6:1 E:T ratio (n=6 wells/time point). T cells were harvested, counted and replated on fresh tumor cells as indicated (●) F) IE9-mp1-hFRa target cell counts (n=8-9 ROI/condition) following final 72 hr co-culture with FR-B 2/7, FR-B 2/15, or Unarmed Control T cells (serial co-culture stress test). G) Survival of IE9-mp1-hFRa tumor bearing mice treated locoregionally with FR-B T cells or Unarmed Control T cells by IP injection (n=10/group). Data presented as mean±SEM. Data in A-D) & F) is from one representative experiment. Data in E) & G) compiled from 2 independent experiments. Data in C & E) Two-way ANOVA, D & F) One-way ANOVA, G) Long-rank Test, * p<0.05, ** p<0.01, * ** p<0.001, **** p<0.0001.

FIG. 5: IL-2/IL-15 preconditioning increases persistence of stem-like FR-B CD8+ TALs in the OC peritoneal TME: Representative FACs plots quantifying the frequency of FR-B CD8+ T cells (GFP+) in the blood & tumor (A) or peritoneal TME (B, left) 5 days post ACT. (B, right) Graphical representation of FR-B CD8+ TAL frequency (n=8-9/group). C-E) Representative FACs plots showing the frequency of Ki67+(C), CD39+/CD69+(D) and TCF-1+(E) FR-B CD8+ TALs following 2/7 or 2/15 preconditioning. F) Volcano plot of differentially expressed genes between FR-B 2/7 and FR-B 2/15 CD8+ TALs (n=2-3/group). G) Pathway enrichment analysis for data shown in F). Data presented as mean±SEM. Data in B) compiled from 2 independent experiments. Data in B) Unpaired t test (two-tailed), ** p<0.01

FIG. 6: Proposed Mechanism of action for durable antitumor immunity following locoregional infusion of FR-B T cells in Ovarian Cancer: Left) FR-B 2/7 T cells have robust effector function, but limited persistence in either the peritoneal TME or solid tumor lesions, leading to short-term BiTE-mediated antitumor immunity and therapeutic failure. Right) FR-B 2/15 T cells develop a stem-like phenotype and effectively persist with high frequency within the extratumoral peritoneal TME to functionally direct antitumor immune responses, resulting in prolonged BiTE activity and durable antitumor immunity.

FIG. 7: Generation of FRα-targeted FR-Bh BiTE and Functional testing of FR-Bh T cells: A) Graphical depiction of FR-Bh retroviral construct configuration (left) and specific binding of FR-Bh to FRα+ cells and T cells (right). CONT-ENG (which lacks human CD3 binding) and Control Supernatant (Cont Sup) containing no BiTEs/Engagers, as well as CD19+B cells were included as staining controls. The EAAAK sequence on FIG. 7A is SEQ ID NO:9. The full sequence (GEAAAKEAAAKEAAAK) is SEQ ID NO:8. The GSTSGSGKSSEGKG is SEQ ID NO:13. The Leader sequence MNSGLQLVFFVLTLKGIQ is SEQ ID NO:14. The SGSGHHHHHH (with the His tag shown as His6) is SEQ ID NO:15. The RAKRSGSG (P2A) sequence is SEQ ID NO:16. B) Surface FRα on cancer cell lines measured by flow cytometry (▪) compared to FMO Controls (□). K562 cells included as a 20 negative control C) SKOV-6 Target cell number (left, n=2 wells/condition) and IFN-γ production (right, n=3/well) measured in a transwell assay. SKOV-6 target cells were plated in the lower chamber, with UTD or BiTE-producing T cells added to the upper/lower chambers as indicated. D) SKOV-6 tumor growth following therapeutic delivery of CONT-ENG or FR-Bh T cells, delivered by split dose injection (IV/Intratumoral delivery, n=3/group). Data presented as mean±SEM. Data in C) is from one representative experiment and data in D) is from one experiment. C) One-way ANOVA, D) Two-way ANOVA, ** p<0.01, **** p<0.0001.

FIG. 8: Development of a translational model to test FR-Bh T cells against ovarian cancer patient specimens: A) Schematic diagram illustrating experimental setup for OC patient co-cultures B) Representative FACs plots demonstrating surface FRα levels in Frα^{lo}(left) and Frα^{hi} (right) OC patients. C) FRα+(□) and FRα−(▪) tumor cell number and corresponding IFN-γ production (●) from 48 hr OC patient co-cultures following addition of CONT-ENG or FR-Bh T cells for patients not shown in the main text figures. Baseline tumor cell number and IFN-γ (co-cultures containing endogenous TALs only) shown for comparison.

FIG. 9: Gating strategy and activation status of T cell subsets in OC patient co-cultures following addition of FR-Bh or Cont-ENG T cells: A) Gating strategy used to subset endogenous (GFP− Cell Trace−), Bystander (GFP− Cell Trace+), and Transduced (GFP+ Cell Trace+) T cells in OC patient co-cultures B) FACs plots and graphical data demonstrating activation of Transduced CD4+ T cells in FRα+(right, upper, n=7) and Frα^{lo} (right, lower, n=3) OC patients C) Surface markers of activation measured on FR-Bh CD8+ T cells cultured alone for the 48 hr co-culture period, demonstrating limited activation in the absence of target antigen D & E) Activation of bystander CD8+ T cells (D) and endogenous CD4+ TALs (E) from FRα+OC patient co-cultures. Data in B & D) Unpaired t test (two-tailed), * p<0.05, ** p<0.01

FIG. 10: Generation FRα-targeted FR-B BiTE for use in preclinical mouse models and therapeutic testing of FR-B T cells: A) Graphical depiction of FR-B retroviral construct configuration. For A), the EAAAK is SEQ ID NO:7. The full sequence is SEQ ID NO:8. The GSTSGSGKSSEGKG sequence is SEQ ID NO:13. The SGSGHHHHHH (with the His tag shown as His6) is SEQ ID NO:15. B) Specific binding of FR-B to IE9-mp1-hFRa cells and mouse CD8+ T cells. FR-Bh (which lacks mouse CD3 binding), Control Supernatant (Cont Sup) containing no BiTEs, and CD19+B cells were included as staining controls. C) Experimental Design (left), tumor growth (middle), and survival of Pan02-hFRa tumor bearing mice treated locoregionally with FR-B T cells or Unarmed Control T cells by IP injection (n=6/group). D) Mice were lymphodepleted by delivery of 5Gy TBI immediately prior to tumor implantation, with delivery of FR-B T cells 5 days later. Tumor progression was tracked based on accumulation of peritoneal ascites, measured as increased abdominal circumference (n=5/group). E) Representative FACs plots demonstrating limited persistence of FR-B CD8+ or CD4+ T cells in either the tumor or ascites at disease endpoint. Data presented as mean±SEM. Data in C) is from one experiment and data in D) is from one representative experiment. C) Two-way ANOVA (left) and Log-rank Test (right), D) Two-way ANOVA, **** p<0.0001.

FIG. 11: Graphical depiction of serial stress test co-culture system to model persistent and repeated antigen stimulation of FR-B T cells.

FIG. 12: IL-2/IL-15 preconditioning of FR-B T cells promotes improved persistence of stem-like CD8+FR-B TALs within the peritoneal OC TME: A) % of CD4+FR-B T cells in blood, peritoneal wash, and tumor 5 days post ACT (n=3-4/group) B) Frequency of CD8+FR-B T cells in blood & tumor 5 days post ACT (n=3-4/group C) Representative FACs plots demonstrating activation (CD69) and proliferation (Ki67) of Endogenous/Bystander CD8+ TILs (GFP−) 5 days post ACT D) % CD8+FR-B TALs of total CD45+ Immune infiltrate in the OC peritoneal TME (n=8-9/group) E-G) Quantitative analysis of % Ki67+(E) CD39/CD69+(F) and TCF-1+(G) CD8+FR-B TALs 5 days post ACT as shown in FIG. 5 (n=4/group). Data presented as mean±SEM. Data in D) compiled from 2 independent experiments. Data in D-G) Unpaired t test (two-tailed) * p<0.05.

FIG. 13: Transcriptional profiling of CD8+FR-B TALs: A) Normalized expression of key effector and checkpoint genes measured by RNAseq for CD8+FR-B 2/7 and 2/15 TALs (n=2-3/group) B) Hierarchical clustering of differentially expressed genes between CD8+FR-B 2/7 and 2/15 TALs (n=2-3/group)

FIG. 14: Supplemental Table 1.

FIG. 15: Supplemental Table 2.

FIG. 16: Data showing survival of IE9-mp1-hFRa tumor bearing mice treated locoregionally with the indicated number of doses of FR-B 2/15 T cells or Unarmed Control T cells by IP injection (n=8-10/group).

### DETAILED DESCRIPTION

Unless defined otherwise herein, all technical and scientific terms used in this disclosure have the same meaning as commonly understood by one of ordinary skill in the art to which this disclosure pertains.

The following abbreviations are used in this disclosure:
ACT: Adoptive T cell Transfer
ANOVA: Analysis of variance
BiTEs: Bispecific T cell engagers
BiTE-T cells: BiTE-secreting T cells
CAR: Chimeric Antigen Receptor
cDMEM: Complete DMEM (media)
CONT-ENG: Control engager
CTV: CellTrace Violet
cRPMI: Complete RPMI (media)
E:T: Effector to target ratio
FACs: Fluorescence activated cell sorting
FBS: Fetal bovine serum
FRα: Folate receptor alpha
FR-B T cells: Folate receptor alpha bispecific T cell engager-secreting T cells (mouse)
FR-Bh T cells: Folate receptor alpha bispecific T cell engager-secreting T cells (human)
GFP: Green fluorescent protein
GM-CSF: Granulocyte-macrophage colony-stimulating factor
Gyn Onc: Gynecologic oncology
hFRα: Human Folate receptor alpha
hrs: Hours
IFN-γ: Interferon gamma
IHC: Immunohistochemistry
IL-2, 5, 6, 7, 13, 15: Interleukin 2, 5, 6, 7, 13, 15
IP: Intraperitoneal
IU: International unit
Luc/GFP: Luciferase-Green fluorescent protein fusion protein
MIP-1α/β: Macrophage inflammatory protein 1 alpha and beta
MIRV: mirvetuximab soravtansine
ml: Milliliter
mM: Millimolar
OC: Ovarian cancer
PBS: Phosphate buffered saline
PDX: Patient-derived xenograft
RNAseq: RNA sequencing
scFv: Single chain variable fragment
SQ: Subcutaneous
TALs: Tumor-associated lymphocytes
TBI: Total Body Irradiation
TCR: T cell receptor
TILs: Tumor infiltrating T cells
TME: Tumor microenvironment
Tregs: Regulatory T cells
TSCM: Stem cell memory T cells
UTD: Untransduced

Every numerical range given throughout this specification includes its upper and lower values, as well as every narrower numerical range that falls within it, as if such narrower numerical ranges were all expressly written herein.

The disclosure includes all polynucleotide and amino acid sequences described herein. Amino acids of all protein sequences and all polynucleotide sequences encoding them are also included, including but not limited to sequences included by way of sequence alignments. Sequences of from 80.00%-99.99% identical to any sequence (amino acids and nucleotide sequences) of this disclosure are included. The disclosure includes all amino acid and polynucleotide sequences that are identified herein by way of a database entry as the sequences exist in the database as of the effective filing date of this application.

The disclosure includes all compositions, results, and method steps alone and in combination, and described herein and as depicted in the Figures.

As discussed above, the disclosure provides binding partners provided as bispecific T cell engagers (BiTEs). Thus, the binding partners are in certain examples are multivalent. In embodiments, leukocytes, including but not necessarily limited to T cells, express a BiTE.

In one approach the present disclosure combines BiTE-based technologies and the therapeutic approach of targeting FRα to develop a novel ACT approach for OC and other cancers that utilizes engineered FRα-targeted BiTE-T cells (referred to from time to time herein as FR-Bh T cells for human T cells, and FR-B T cells for mouse T cells). As demonstrated in the Examples and Figures of this disclosure, FR-Bh T cells were highly effective against both FRα+OC patient samples and in immunocompetent preclinical tumor models. Moreover, mechanistic studies revealed that improved therapeutic efficacy was accompanied by preferential accumulation of less differentiated stem-like FR-B T cells in the extratumoral peritoneal OC TME over solid tumor lesions. This indicates that FR-B T cells in remote locations can promote tumor destruction in OC (by secreting BiTEs and engaging endogenous T cells) without a requirement for direct accumulation in solid tumors. The disclosure is therefore expected to be suitable for use as an ACT therapy used to treat solid tumors, including but not necessarily limited to OC, where limited tumor reactivity from endogenous T cells can create therapeutic challenges.

In embodiments, binding partners of this disclosure may comprise linking sequences. Suitable amino acid linkers may be mainly composed of relatively small, neutral amino acids, such as glycine, serine, and alanine, and can include multiple copies of a sequence enriched in glycine and serine. In specific and non-limiting embodiments, the linker comprises 3, 4, 5, 6, 7, 8, 9, 10, 11, 12, 13, 14, 15, 16, 17, 18, 19, and 20 amino acids. In an example, the linker may be the glycine-serine-alanine linker G₄SA₃ (SEQ ID NO:18) or a glycine-serine linker (G₄S)₄ (SEQ ID NO:19) linker. Representative examples of linking sequences are provided below in the described sequences.

In embodiments, such as for proteins that are produced as a fusion protein, a peptide linker may be used, and may comprise a cleavable or non-cleavable linker. In embodiments, the peptide linker comprises any self-cleaving signal. In embodiments, the self-cleaving signal may be present in the same open reading frame (ORF) as the ORF that encodes the binding partner. A self-cleaving amino acid sequence is typically about 18-22 amino acids long. Any suitable sequence can be used, non-limiting examples of which include: T2A, P2A, E2A, and F2A, the sequences of which are known in the art.

In embodiments, a binding partner may include a secretion signal. The present disclosure therefore provides modified cells, such as T cells, that secrete a described binding partner. For secretion, any suitable secretion signal can be used and many are known in the art. In non-limiting embodiments, the secretion signal comprises MALPVTALLLPLALLLHA (SEQ ID NO:1), METDTLLLWVLLLWVPGSTG (SEQ ID NO:2), or MGWSCIILFLVATATGVHSD (SEQ ID NO:3) or GEAAAKEAAAKEAAAK (SEQ ID NO:8). Representative examples of proteins produced and secreted by modified cells according to this disclosure are described further below. For amino acid sequences of this disclosure that include amino acids that comprise purification or protein production tags, including but not limited to HIS tags, the disclosure includes the proviso that the sequences of any described tag may be excluded from the claimed amino acid sequences. Further, where linker sequences are identified, any suitable linker sequence may be substituted for the described sequence. Linker sequences and/or purification tag sequences may be excluded from sequence similarity values described herein. Any binding partner described herein may be fully or partially humanized.

For therapeutic approaches, in certain embodiments, a described binding partner may be delivered as mRNA or DNA polynucleotides that encode the binding partner. It is considered that administering a DNA or RNA encoding any binding partner described herein is also a method of delivering such binding partners to an individual or to one or more cells, provided the DNA is transcribed and the mRNA is translated, and/or the RNA itself is delivered and translated. Methods of delivering DNA and RNAs encoding proteins are known in the art and can be adapted to deliver the binding partners, given the benefit of the present disclosure. In embodiments, one or more expression vectors are used and comprise viral vectors. Thus, in embodiments, a viral expression vector is used. Viral expression vectors may be used as naked polynucleotides, or may comprises any of viral particles, including but not limited to defective interfering particles or other replication defective viral constructs, and virus-like particles. In embodiments, the expression vector comprises a modified viral polynucleotide, such as from an adenovirus, a herpesvirus, or a retroviral vector. In embodiments, the retroviral vector is adapted from a murine Moloney leukemia virus (MLV) or a lentiviral vector may be used, such as a lentiviral vector adapted from human immunodeficiency virus type 1 (HIV-1). Representative demonstrations of the disclosure are provided and use mouse stem cell virus (MSCV)-based retroviral vector to produce retrovirus in Platinum-E (PLAT-E) virus packaging cells for mouse T cell transduction or PG13 virus packaging cells for human T cell transduction.

In alternative embodiments, a recombinant adeno-associated virus (AAV) vector may be used. In certain embodiments, the expression vector is a self-complementary adeno-associated virus (scAAV).

In embodiments, cells modified according to this disclosure include mature T cells, or their progenitor cells such hematopoietic stem cells or any other time of T cell progenitor cells. The disclosure includes progeny of progenitor cells. Thus, in embodiments, cells that are modified to express any binding partner described herein include but are not necessarily limited CD4+ T cells, CD8+ T cells, Natural Killer T cells, γδ T cells, and cells that are progenitors of T cells, such as hematopoietic stem cells or other lymphoid progenitor cells, immature thymocytes (double-negative CD4-CD8-) cells, or double-positive thymocytes (CD4+CD8+). In embodiments, the progenitor cells comprise markers, such as CD34, CD117 (c-kit) and CD90 (Thy-1). In embodiments, a population of human peripheral blood mononuclear cells are modified using the described polynucleotides.

In embodiments, a polynucleotide that encodes a described binding partner selectively hybridizes to a polynucleotide encoding at least one protein that is a component of a binding partner, including but not limited to a heavy chain CDR1, CDR2, and CDR3 of any described binding partner. In embodiments, the polynucleotide selectively hybridizes to a polynucleotide encoding a light chain CDR1, CDR2 and CDR3 of any described binding partner. In embodiments, the polynucleotide selectively hybridizes to a polynucleotide encoding CDR1, CDR2 and CDR3 of a heavy and light chain of any described binding partner.

Pharmaceutical formulations containing binding partners are included in the disclosure, and can be prepared by mixing them with one or more pharmaceutically acceptable carriers. Pharmaceutically acceptable carriers include solvents, dispersion media, isotonic agents and the like. The carrier can be liquid, semi-solid, e.g. pastes, or solid carriers. Examples of carriers include water, saline solutions or other buffers (such as phosphate, citrate buffers), oil, alcohol, proteins (such as serum albumin, gelatin), carbohydrates (such as monosaccharides, disaccharides, and other carbohydrates including glucose, sucrose, trehalose, mannose, mannitol, sorbitol or dextrins), gel, lipids, liposomes, resins, porous matrices, binders, fillers, coatings, stabilizers, preservatives, liposomes, antioxidants, chelating agents such as EDTA; salt forming counter-ions such as sodium; non-ionic surfactants such as TWEEN, PLURONICS or polyethylene glycol (PEG), or combinations thereof.

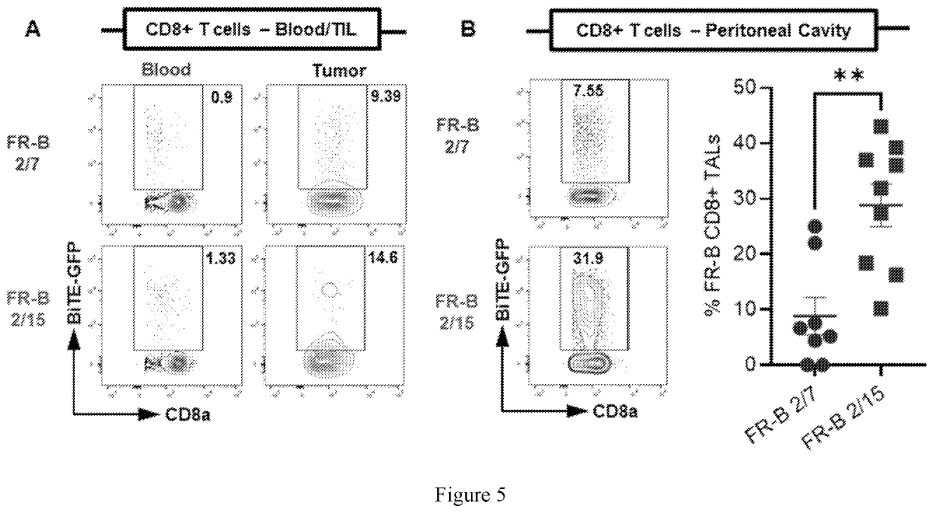

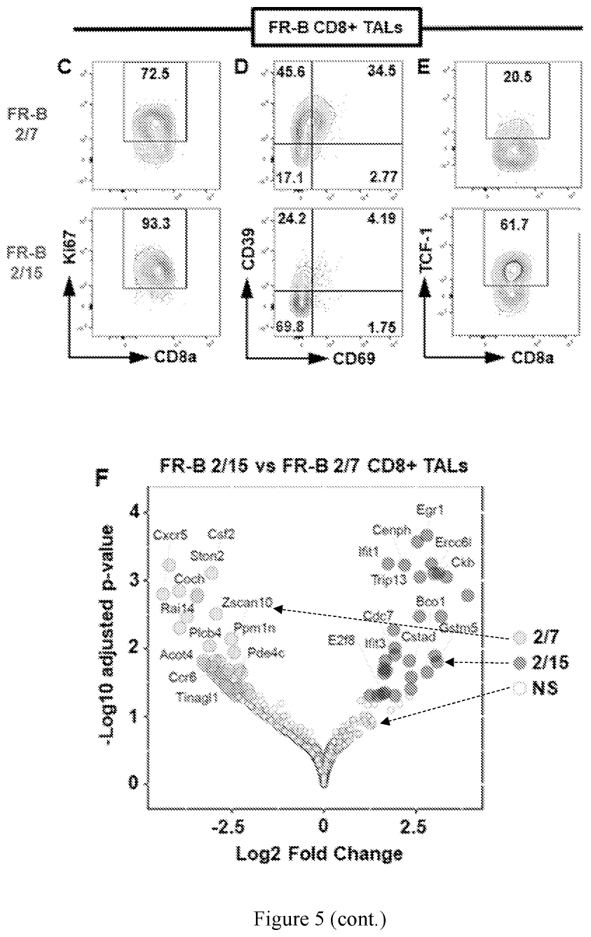

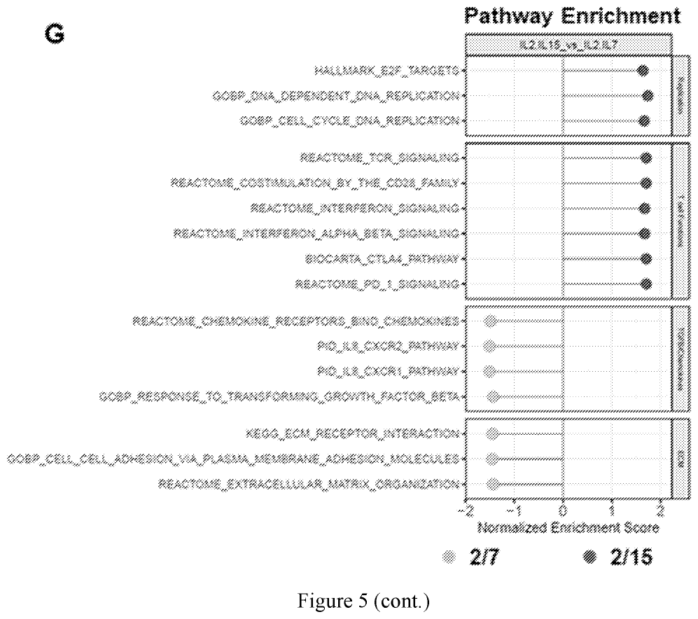

In embodiments, an effective amount of T cells expressing a described binding partner is administered to an individual in need thereof. In embodiments, an effective amount is an amount that reduces one or more signs or symptoms of a disease and/or reduces the severity of the disease. An effective amount may also inhibit or prevent the onset of a disease or a disease relapse. A precise dosage can be selected by the individual physician in view of the patient to be treated. Dosage and administration can be adjusted to provide sufficient levels of binding partner to maintain the desired effect. Additional factors that may be taken into account include the severity and type of the disease state, age, weight and gender of the patient, desired duration of treatment, method of administration, time and frequency of administration, drug combination(s), reaction sensitivities, and/or tolerance/response to therapy. In embodiments an effective amount is an amount of modified T cells that express and secrete a binding partner and produces a therapeutic effect without of the modified T cells in a solid tumor that is 1-20% of the total T cells present in the solid tumor. In embodiment the engineered T cells are about 10% of the total T cells in a solid tumor (see, for example, FIG. 5).

The described binding partners and T cells that express and secrete the binding partners can be administered directly or provided as pharmaceutical compositions and administered to an individual in need thereof using any suitable route, examples of which include intravenous, intramuscular, intraperitoneal, intracerobrospinal, subcutaneous, intra-articular, intrasynovial, oral, topical, or inhalation routes, depending on the particular condition being treated. Intra-tumor injections may also be used. The compositions may be administered parenterally or enterically. The compositions may be introduced as a single administration or as multiple administrations or may be introduced in a continuous manner over a period of time, non-limiting examples of which are demonstrated herein.

In embodiments, the described compositions are suitable for use in humans. The disclosure also includes the described constructs that are suitable for use in syngeneic immunocompetent mouse models.

In embodiments, the individual in need of a composition of this disclosure has been diagnosed with or is suspected of having cancer. In embodiments, the cancer is a solid or liquid tumor. In embodiments, the cancer is renal cell carcinoma, breast cancer, prostate cancer, pancreatic cancer, lung cancer, liver cancer, ovarian cancer, cervical cancer, colon cancer, esophageal cancer, glioma, glioblastoma, or another brain cancer, stomach cancer, bladder cancer, testicular cancer, head and neck cancer, melanoma or another skin cancer, any sarcoma, including but not limited to fibrosarcoma, angiosarcoma, adenocarcinoma, and rhabdomyosarcoma, and any blood cancer, including all types of leukemia, lymphoma, and myeloma.

In embodiments, administering a described binding partner, such as by way of administering T cells that are modified to secrete the described binding partner, exhibits an improved activity relative to a control. In an embodiment, the control comprises administration of a BiTE without using T cells that secrete the BiTE. In embodiment, the control comprises a BiTE that is secreted by a cell that is not a T cell.

The described T cells that express the described binding partners can be combined with any other therapeutic agent, non-limiting examples of which include conventional chemotherapeutic agents, and immune checkpoint inhibitors, the latter of which are known in the art, and target CTLA4, PD-1, or PD-L1. Thus, the disclosure includes combination therapy using one or more described binding partners and any of CTLA-4 inhibitors, PD-1 inhibitors and PD-L1 inhibitors. As non-limiting examples, anti-PD-1 agents include Pembrolizumab and Nivolumab. Anti-PD-L1 examples include Avelumab and Atezolizumab. An anti-CTLA-4 example is Ipilimumab. The binding partners may also be combined with any other form of adoptive immunotherapy. The modified T cells may be used in autologous or allogenic therapies.

The disclosure includes the described expression vectors that encode the BiTEs, and all methods of making T cells that are described herein and by way of the figures. In embodiments, the disclosure provides for obtaining T cells from an individual and subjecting the T cells to cytokine treatment to prepare the T cells for use as an adoptive immunotherapy, and modifying the T cells to express the described BiTEs. The T cells may be modified to express the described BiTEs before, during or after cytokine treatment, but the cytokine treatment is before perfusion into an individual. In embodiments, the cytokine treatment comprises repeated IL-15 treatments. In embodiments, the cytokine treatment comprises IL-2 +IL-7, or IL-2+IL-15. In embodiments, substituting IL-15 in place of IL-7 enhances properties of the T cells, such as expansion/persistence following infusion, a stem-like phenotype, and improved tumor control, as illustrated in the Figures.

As is known in the art, BiTEs incorporate single-chain variable fragments (scFvs) and are composed of a tumor-targeted scFv providing tumor antigen target specificity, linked in tandem to a T cell specific scFv, which provides T cell activation (typically an anti-CD3 scFv).

The disclosure provides non-limiting examples of embodiments that are illustrated in the Figures and described in the Examples below. Data produced and described in the accompanying figures showing production of modified cells and results from use of said cells that express and secrete the BiTEs were obtained using polynucleotides that encode the following sequences:

Anti-mouse CD3e scFV
Clone: 145-2C11
Format: VH/VL
Standard G4S Linker (bold)
(SEQ ID NO: 4)
EVQLVESGGGLVQPGKSLKLSCEASGFTFSGYGMHWVRQAPGRGLESVAY
ITSSSINIKYADAVKGRFTVSRDNAKNLLFLQMNILKSEDTAMYYCARFD
WDKNYWGQGTMVTVSSGGGGSGGGGSGGGGSDIQMTQSPSSLPASLGDRV
TINCQASQDISNYLNWYQQKPGKAPKLLIYYTNKLADGVPSRFSGSGSGR
DSSFTISSLESEDIGSYYCQQYYNYPWTFGPGTKLEIK
Anti-human CD3e scFv
Clone: UCHT1
Format: VL/VH
Standard G4S Linker (bold)
(SEQ ID NO: 5)
MDIQMTQTTSSLSASLGDRVTISCRASQDIRNYLNWYQQKPDGTVKLLIY
YTSRLHSGVPSKFSGSGSGTDYSLTISNLEQEDIATYFCQQGNTLPWTFA
GGTKLEIKGGGGSGGGGSGGGGSGGGGSEVQLQQSGPELVKPGASMKISC
KASGYSFTGYTMNWVKQSHGKNLEWMGLINPYKGVSTYNQKFKDKATLTV
DKSSSTAYMELLSLTSEDSAVYYCARSGYYGDSDWYFDVWGQGTTLTVFS
Anti-human FRα scFv
Clone: MOv19
Format: VL/VH
Non-standard (referred to as 212)
Linker (bold)
(SEQ ID NO: 6)
DIELTQSPASLAVSLGQRAIISCKASQSVSFAGTSLMHWYHQKPGQQPKL
LIYRASNLEAGVPTRFSGSGSKTDFTLNIHPVEEEDAATYYCQQSREYPY
TFGGGTKLEIKGSTSGSGKSSEGKGQVQLQQSGAELVKPGASVKISCKAS
GYSFTGYFMNWVKQSHGKSLEWIGRIHPYDGDTFYNQNFKDKATLTVDKS
SNTAHMELLSLTSEDFAVYYCTRYDGSRAMDYWGQGTTVTVS
BiTE Sequences
Mouse FRα targeted BiTE (mCD3e scFv x hFRα
scFv); FR-B
mCD3e scFV = 145-2C11 Clone
hFRα scFV = Mov19 Clone
*Chimeric BiTE = binds human target antigen and
mouse CD3 for use in syngeneic immunocompetent
mouse models
*Signal Sequence (secretion signal) at
N terminus in italics
(SEQ ID NO: 8)
GEAAAKEAAAKEAAAK
linker sequence; Rigid and long linker sequence
between scFv was tested in parallel with multiple
linkers of varying length/flexibility and was
found to result in optimal antigen binding by both
CD3e and FRα targeted scFv's in the current
format and resulted in optimal in vivo activity
*Short linker + 6x His Tag at C terminus
(SEQ ID NO: 7)
MNSGLQLVFFVLTLKGIQGEVQLVESGGGLVQPGKSLKLSCEASGFTFSG
YGMHWVRQAPGRGLESVAYITSSSINIKYADAVKGRFTVSRDNAKNLLFL
QMNILKSEDTAMYYCARFDWDKNYWGQGTMVTVSSGGGGSGGGGSGGGGS
DIQMTQSPSSLPASLGDRVTINCQASQDISNYLNWYQQKPGKAPKLLIYY
TNKLADGVPSRFSGSGSGRDSSFTISSLESEDIGSYYCQQYYNYPWTFGP
GTKLEIKGEAAAKEAAAKEAAAKDIELTQSPASLAVSLGQRAIISCKASQ
SVSFAGTSLMHWYHQKPGQQPKLLIYRASNLEAGVPTRFSGSGSKTDFTL
NIHPVEEEDAATYYCQQSREYPYTFGGGTKLEIKGSTSGSGKSSEGKGQV
QLQQSGAELVKPGASVKISCKASGYSFTGYFMNWVKQSHGKSLEWIGRIH
PYDGDTFYNQNFKDKATLTVDKSSNTAHMELLSLTSEDFAVYYCTRYDGS
RAMDYWGQGTTVTVSSGSGHHHHHH
Human FRα targeted BiTE (hCD3e scFV x
hFRα scFv)
hCD3e scFv = UCHT1 clone
hFRa scFv = Mov19 clone
*Signal Sequence at N terminus in italics
(SEQ ID NO: 8)
GEAAAKEAAAKEAAAK;
(SEQ ID NO: 9)
G(EAAAK)
3 linker sequence:
Same linker sequence as used in mouse FRα
targeted BiTE
Short linker + 6x His Tag at C terminus
(SEQ ID NO: 10)
MNSGLQLVFFVLTLKGIQGMDIQMTQTTSSLSASLGDRVTISCRASQDIR
NYLNWYQQKPDGTVKLLIYYTSRLHSGVPSKFSGSGSGTDYSLTISNLEQ
EDIATYFCQQGNTLPWTFAGGTKLEIKGGGGSGGGGSGGGGSGGGGSEVQ
LQQSGPELVKPGASMKISCKASGYSFTGYTMNWVKQSHGKNLEWMGLINP
YKGVSTYNQKFKDKATLTVDKSSSTAYMELLSLTSEDSAVYYCARSGYYG
DSDWYFDVWGQGTTLTVFSGEAAAKEAAAKEAAAKDIELTQSPASLAVSL
GQRAIISCKASQSVSFAGTSLMHWYHQKPGQQPKLLIYRASNLEAGVPTR
FSGSGSKTDFTLNIHPVEEEDAATYYCQQSREYPYTFGGGTKLEIKGSTS
GSGKSSEGKGQVQLQQSGAELVKPGASVKISCKASGYSFTGYFMNWVKQS
HGKSLEWIGRIHPYDGDTFYNQNFKDKATLTVDKSSNTAHMELLSLTSED
FAVYYCTRYDGSRAMDYWGQGTTVTVSSGSGHHHHHH. The dis-
closure includes use of this construct with amino
acids 1-531 only.

For use in humans, in an embodiment a binding partner of this disclosure includes a sequence that is an anti-human CD3e scFv as shown in SEQ ID NO:5. In an embodiment a binding partner of this disclosure includes a sequence that is an anti-human FRα scFv sequence as shown in SEQ ID NO:6. In an embodiment a binding partner of this disclosure includes both a sequence that is an anti-human CD3e sequence and a sequence that is an anti-human FRα, such as the sequence shown in SEQ ID NO:10) wherein said sequence includes amino acids 1-531 only and therefore excludes the HIS tag.

### Example 1

The constructs described above were analyzed as described in the following methods.

### Cell Culture

SK-OV-6 (cervical), SK-OV-3 (ovarian), OV167 (ovarian), OVCAR8 (ovarian), OVCAR3 (ovarian), K562 (Leukemia), IE9-mp1 (ovarian), IE9-mp1-hFRa (ovarian), Pan02-hFRa (pancreatic) cancer cell lines were grown in complete RPMI (cRPMI) containing 10% FBS, 25 mM Hepes, 2 mM L-Glutamine, 100 IU/ml Pen/Strep, 1 mM Sodium Pyruvate, 1× Non-Essential Amino Acids, and 0.05 mM β-Mercaptoethanol. 293T, PG13, and PLAT-E cell lines were grown in complete DMEM (cDMEM) containing 10% FBS and 100 IU/ml Pen/Strep. Cell lines were IMPACT tested and/or confirmed mycoplasma negative prior to use.

Generation of hFra-Expressing Cell Lines, BiTE Constructs and Retroviral Vectors

An aggressively growing and immunotherapy resistant IE9-mp1 variant recovered at disease relapse following immunotherapy²⁹ was used to generate the human FRα (hFRa)-expressing IE9-mp1-hFRa cell line using the Sleeping Beauty Transposon system as detailed in herein. As a second murine model, Pan02-hFRa cells was also produced. FR-Bh binds human FRα via a scFv derived from the MOv19 antibody and human CD3ε via a scFv derived from the UCHT1 antibody. FR-B binds human FRα as above and mouse CD3ε via a scFv derived from the 145-2C11 antibody. The design/construction of all engager sequences, production of retroviruses, and testing for engager binding are described below.

### T Cell Activation and Transduction

Human or mouse T cells were activated using anti-CD3ε and anti-CD28 antibodies (Bio X Cell) prior to retroviral transduction. Specific activation/culture conditions and retroviral transduction protocols is described herein.

### In Vitro Co-Cultures

Human or mouse T cells were cultured for no less than 8 days post activation before assay set up. T cells were co-cultured with target cells at the indicated E:T ratios in cRPMI for 24 or 48 hrs. For serial stress test studies involving repeated and prolonged co-culture of mouse T cells with target cells, T cells were harvested, counted, and resuspended in fresh cRPMI+ cytokine support (IL-2+IL-7 or IL-2+IL-15 as indicated) at the start of each new 3-day co-culture period. Additional details are included herein.

### OC Patient Samples and Targeting Using BiTE-T Cells

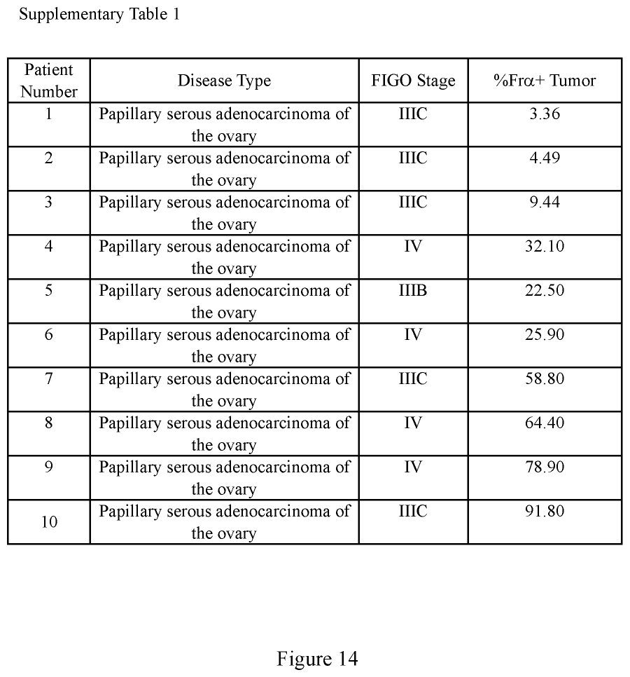

Cryopreserved OC patient ascites samples (Supplemental Table 1 (shown in FIG. 14)) containing both immune cells and tumor cells were obtained from the Roswell Park Gyn Onc Tissue Bank under an approved BDR protocol and were collected from OC patients undergoing care at Roswell Park and processed for banking under approved IRB protocol 1215512. Thawed cells were washed, counted to determine tumor cell number, and plated in 6 well plates at 10⁵ tumor cells/well in cRPMI. Patient samples were cultured ±FR-Bh T cells or T cells secreting a control engager (CONT-ENG T cells) that were pre-labeled with CellTrace Violet and added at a BiTE-T cell: tumor cell ratio of 4:1. OC patient ascites samples ±FR-Bh/CONT-ENG T cells were co-cultured for 48 hrs prior to harvest. Additional details related to these studies have are described herein.

### Preclinical Mouse Models and Therapeutic Delivery of T Cells

FR-Bh T cell evaluation in the SK-OV-6 human xenograft model is described in the supplemental methods. For studies using immunocompetent mice, 6-8-week-old female C57BL/6J mice were purchased from the Jackson Laboratory and housed in the Roswell Park Comparative Oncology Shared Resource (COSR). 5×10⁶IE9-mp1-hFRa cells (IP in 500 μl PBS) or 2×10⁶ Pan02-hFRa (SQ in 100 ul PBS) were injected to establish tumors, with ACT commenced 5 days later. Mice received 8.33×10⁵-3×10⁶ FR-B T cells or an equal number of unarmed control T cells (Luc/GFP transduced or mock transduced) delivered by loco-regional injection (IP or intratumoral delivery for SQ tumors), with timing/dosing as indicated. FR-B T cell accumulation in the blood, peritoneal TME, or solid tumors was assessed 5 days post ACT. Additional details related to in vivo studies, tissue collection, processing, and analysis have been included in the supplemental methods.

### Antibodies and Flow Cytometry Staining/Analysis

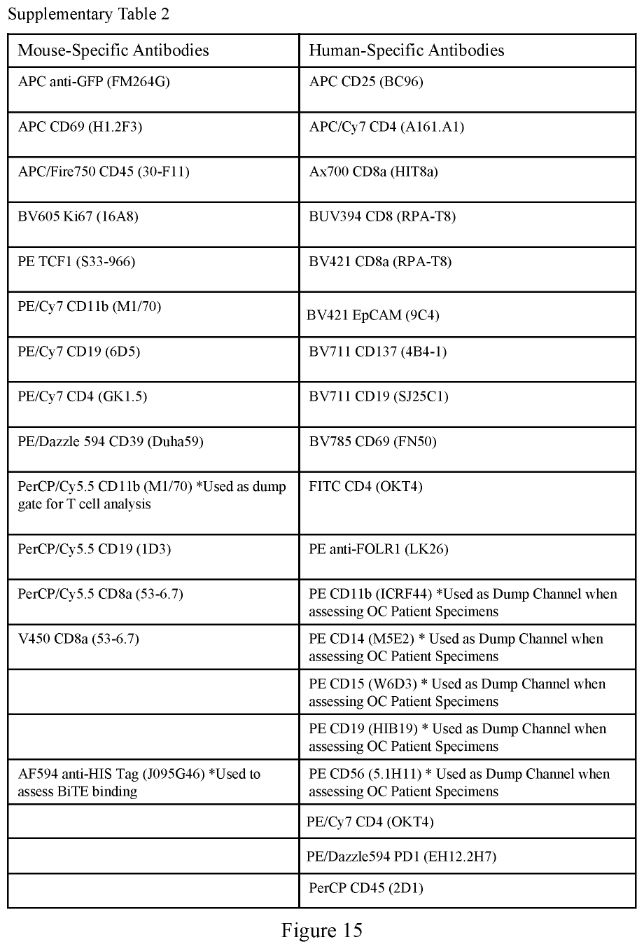

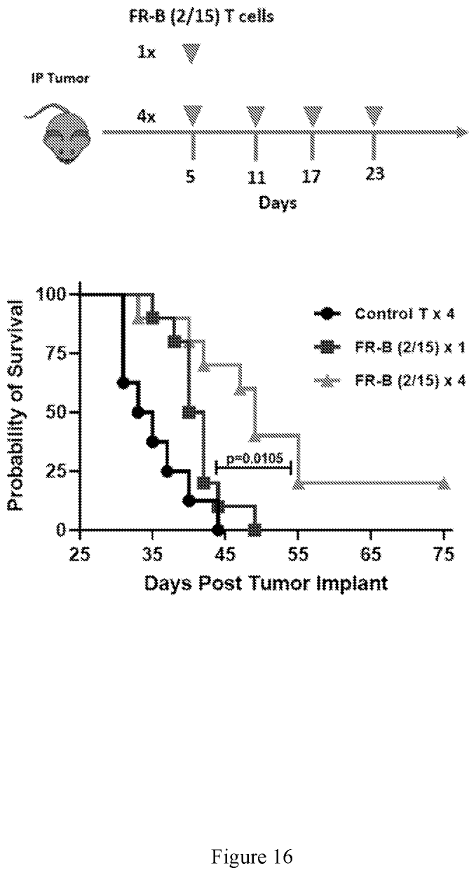

Antibodies for flow cytometry were purchased from BioLegend or BD Biosciences and have been listed in Supplemental Table 2 (shown in FIG. 15). Antibodies were titrated for optimal staining for 30 min at 4° C. in FACs buffer (2% FBS in PBS), BD Horizon™ Brilliant Staining Buffer, or intracellular staining buffer as required. Additional details related to sample staining and analysis have been included in the supplemental methods.

### Statistical Analysis

Two-tailed, unpaired and paired t tests were used to compare data between two groups. One- and two-way Analysis of Variances (ANOVA) were used for data analysis of more than two groups and/or across multiple time points and a Tukey post-test was utilized to determine significant differences between groups. Survival data was compared using a Logrank test. Results were generated using GraphPad Prism software. Differences between means were considered significant at p<0.05: * p<0.05, ** p<0.01, *** p<0.001, **** p<00.0001.

The results in the following Examples were produced using the materials and methods described in Example 1.

### Example 2

Human BiTE-Secreting T Cells have Specificity for FRα+ Cancer Cells and Actively Target OC

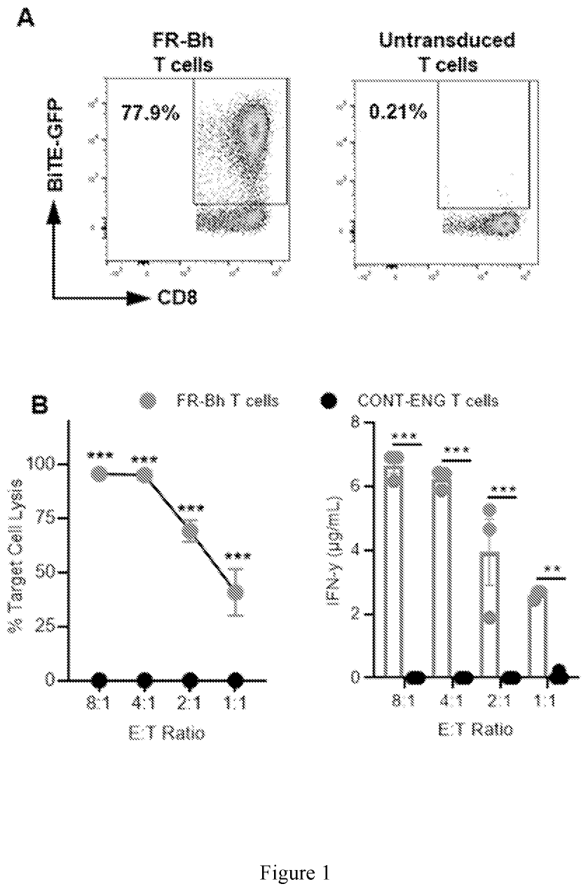

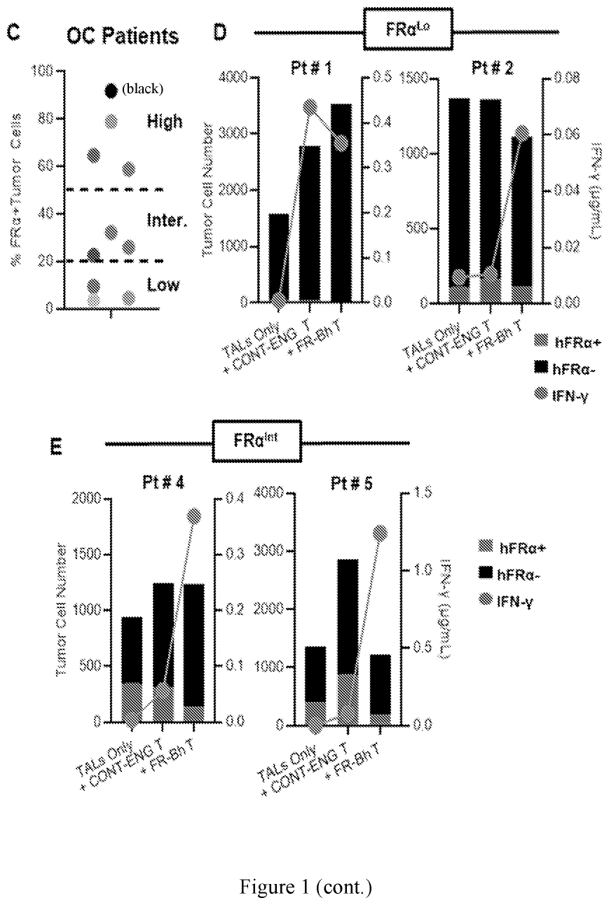

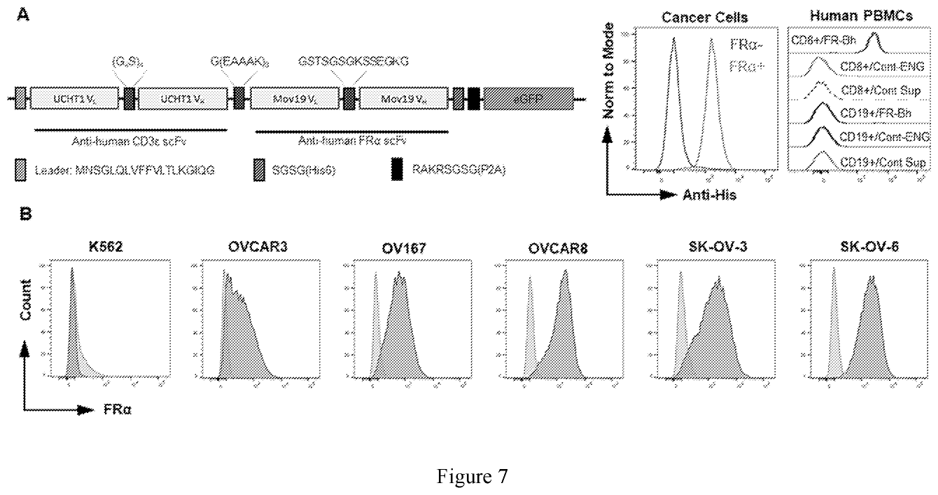

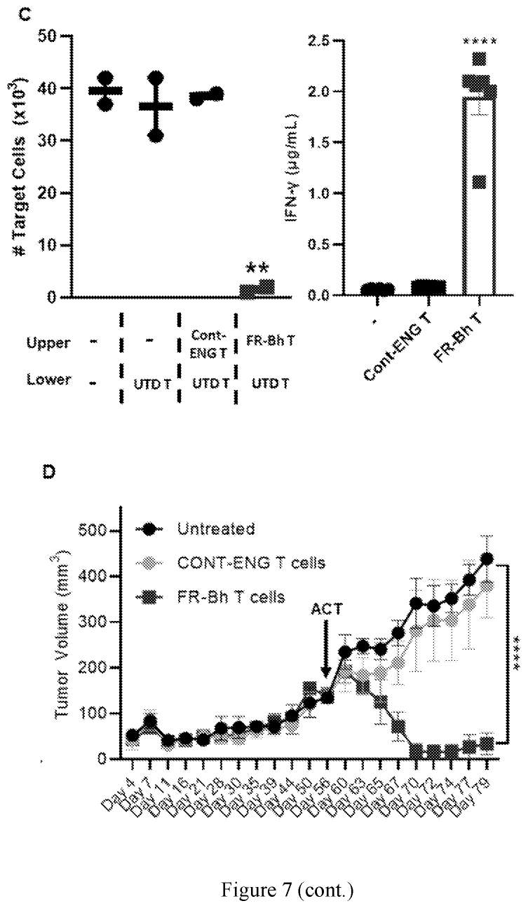

To target FRα+OC, we generated a FRα-specific BiTE by linking a human CD3ε-specific scFv (UCHT1) and a MOV19-derived FRα-specific scFv using optimized linker sequences (7 FIG. 7A, Left Panel). This BiTE, hereafter referred to as FR-Bh, was confirmed to bind FRα+ cancer cells and human T cells (FIG. 7A, Right Panel). BiTE-secreting FR-Bh T cells were efficiently produced using retroviral transduction of activated primary human T cells (FIG. 1A), and in proof-of-concept studies, FR-Bh T cells (but not CONT-ENG T cells) effectively lysed FRα^{hi} SKOV-6 target cells in vitro at even low effector to target (E:T) ratios (FIG. 1B, Left Panel and FIG. 7B). Tumor cell lysis was accompanied by IFN-γ production by FR-Bh T cells (FIG. 1B, Right Panel), consistent with antigen-driven effector function. FR-Bh T cells were confirmed to actively engage bystander T cells (via secreted BiTEs) using a transwell co-culture assay, where FR-Bh T cells plated in the upper chamber led to robust FRα+ target cell killing and effector function by untransduced (UTD) T cells in the lower chamber (FIG. 7C). Therapeutic delivery of FR-Bh T cells to SK-OV-6 tumor-bearing NSG mice produced robust tumor regressions not observed with CONT-ENG T cell infusion (FIG. 7D), confirming therapeutic activity of FR-Bh T cells against growing tumors.

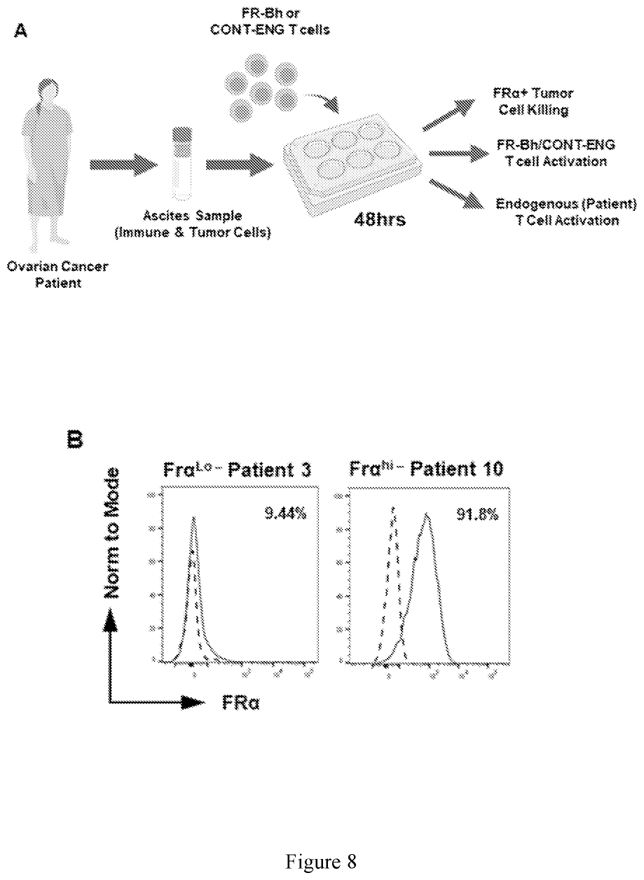

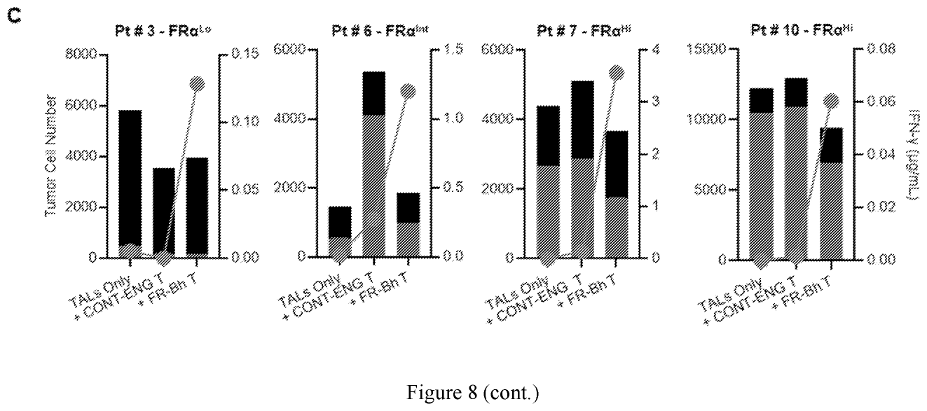

To test if FR-Bh T cells could target clinically-relevant OC, FR-Bh or CONT-ENG T cells were co-cultured at a T cell: tumor cell ratio of 4:1 with OC patient specimens (isolated from peritoneal ascites at the time of surgery, Suppl. Table 1, shown in FIG. 14) containing tumor cells and the patient's own immune cells FIG. 8A). The frequency of FRα+ tumor cells (CD45-EpCAM+ cells) across OC patients was variable, ranging from 3.36% to 91.8% (FIG. 1C, FIG. 8B, & Suppl. Table 1), highlighting the heterogeneity of FRα positivity in OC. Patients with <20% FRα+ tumor cells were considered FRα^{lo} (n=3), 20%-50% FRα+ tumor cells Frα^{Int} (n=3), and >50% FRα+ tumor cells FRα^{h} (n=4), respectively. Following 48 hr co-culture, the FRα+ tumor cell number was reduced in the majority of OC patient co-cultures when FR-Bh T cells were added compared to cultures containing endogenous tumor-associated lymphocytes alone (patient T cells present in ascites; TALs only) or where CONT-ENG T cells were added (FIG. 1D-F & FIG. 8C), which was particularly evident for OC patients with Frα^{Int} or Frα^{hi} tumor cell frequencies. The reduction in FRα+ tumor cells was accompanied by increased IFN-γ production in co-cultures containing FR-Bh T cells for 9/10 patients (FIG. 1D-F & FIG. 8C (lines through bars), with the only exception being a Frα^{lo} OC patient (Patient 1), where the addition of CONT-ENG or FR-Bh T cells produced a similar increase in IFN-γ, possibly due to alloreactivity of the engineered T cells to OC patient cells (FIG. 1D).

To gain insights into the breadth of inflammatory changes driven by FR-Bh T cell therapy against clinical OC specimens, we selected 4 OC patients that responded to FR-Bh T cells from the Frα^{Int} and Frα^{hi} cohorts and broadly analyzed immunological changes in co-cultures containing CONT-ENG T cells or FR-Bh T cells using the Isoplexis Human Adaptive Immune Codeplex Secretome Panel (FIG. 1G). Analysis revealed robust inflammatory changes beyond IFN-γ (Log₁₀ FC=1.83, p=0.0026), including increased production of GM-CSF (Log₁₀ FC=3.21, p=0.0006), Granzyme B (Log₁₀ FC=2.29, p=0.0035), MIP-1α/β(Log₁₀ FC=1.53, p=0.0191 & Log₁₀ FC=1.783, p=0.0213, respectively), as well as upregulation of type-2 cytokines including IL-5 (Log₁₀ FC=1.705, p=0.0039) and a trend towards increased IL-13 (Log₁₀ FC=1.515, p=0.0714) in cultures containing FR-Bh T cells. Of note, the production of multiple factors in co-cultures trended down following the addition of FR-Bh T cells, including IL-6 (Log₁₀ FC=−0.2275, p=0.0571). These data suggest that FR-Bh T cells can be efficiently generated using human T cells for OC targeting and elicit robust antitumor immunity against clinical OC by initiating robust inflammatory responses.

### Example 3

### FR-Bh T Cells Effectively Engage OC Patient T Cells Present in the Peritoneal Tumor Microenvironment

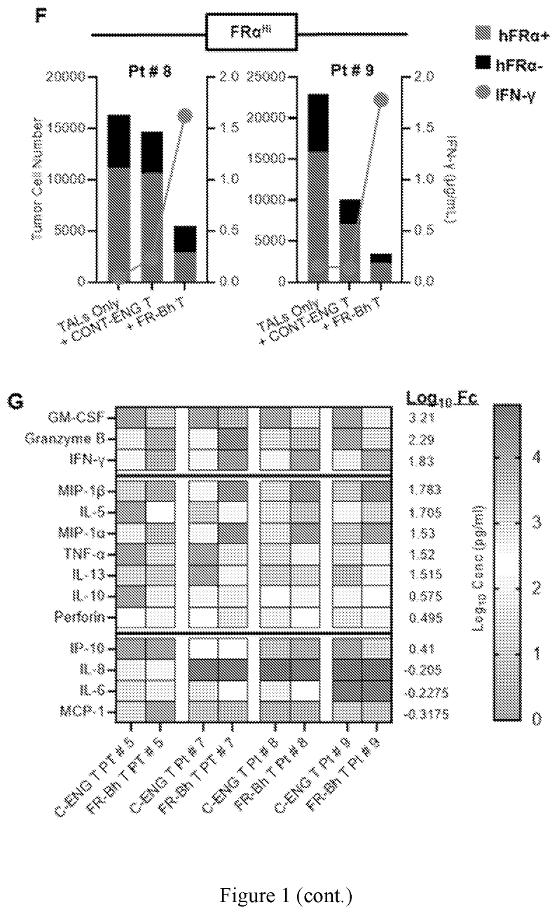

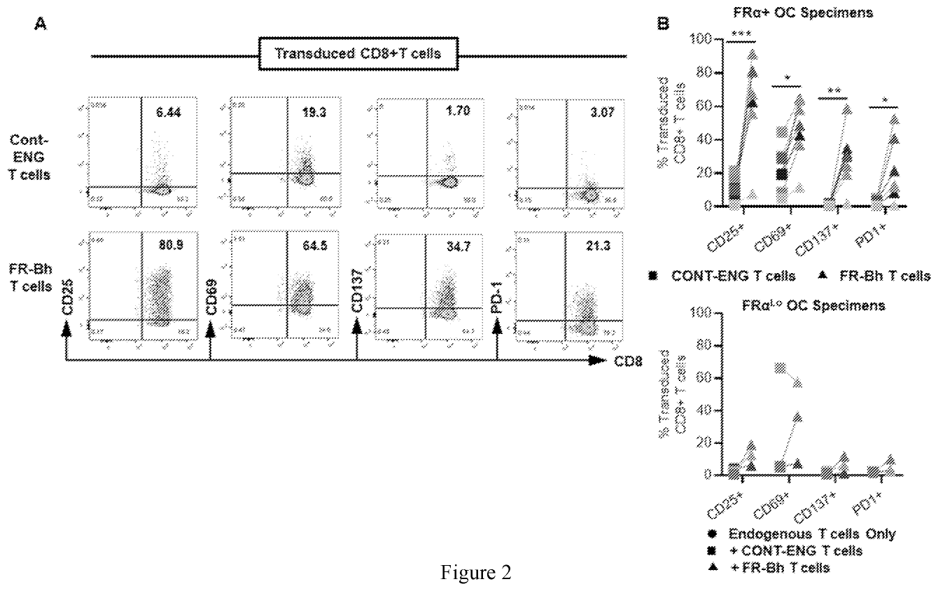

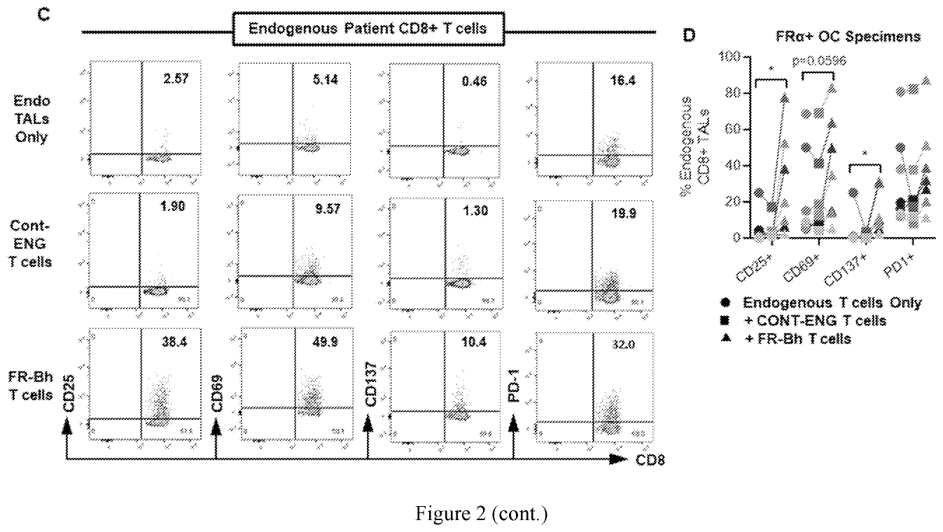

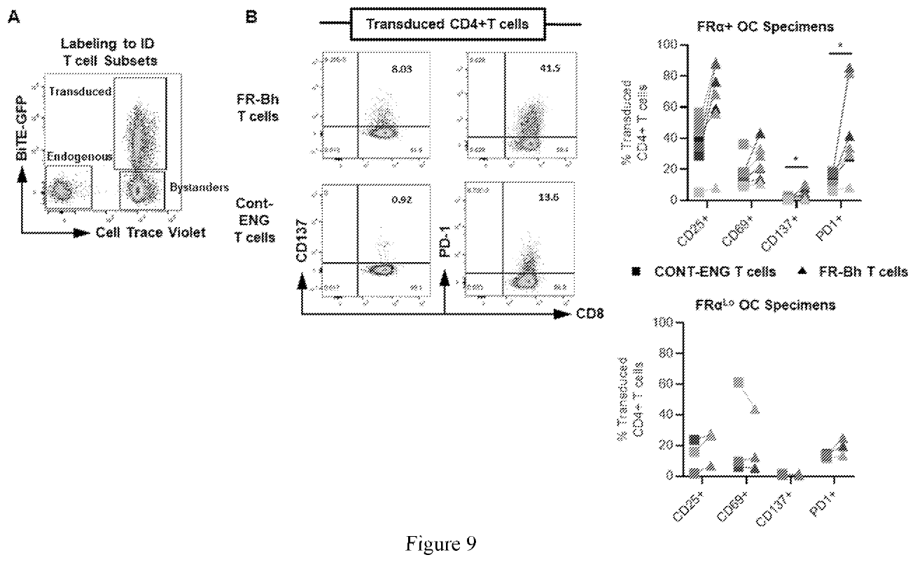

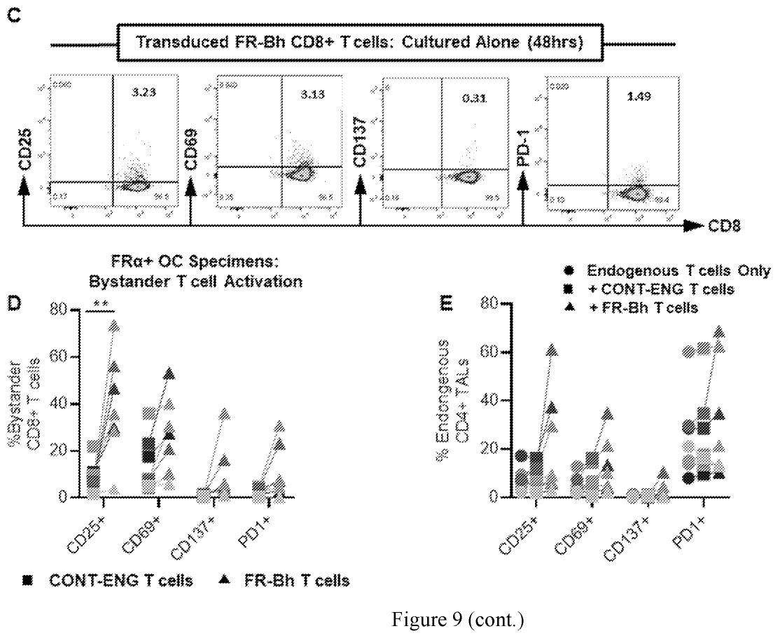

Based on these data, we reasoned that both FR-Bh T cells and endogenous patient T cells present in the OC TME may be actively engaged following delivery of FR-Bh T cells, thereby contributing to the BiTE-driven T cell response. To permit separate interrogation of BiTE-T cell versus host T cell activation in co-cultures containing OC patient specimens, exogenously added T cells (comprised of engineered FR-Bh/CONT-ENG-producing and bystander non-transduced T cells) were labeled with CellTrace Violet (CTV) prior to addition to co-cultures, permitting discrete assessment of transferred [CTV+; transduced (GFP+) and UTD bystander (GFP−) T cells] and endogenous (CTV-GFP−) T cells (FIG. 9A). As we had observed similar inflammatory responses and accompanying reduction in FRα+ tumor cells for Frα^{hi} and Frα^{Int} OC patients following addition of FR-Bh T cells, these patients were grouped as FRα+ patients (>20% FRα+ tumor cells) to evaluate T cell activation and compared to Frα^{lo} OC patients (<20% FRα+ tumor cells) where only modest responses were observed. Following co-culture, CD8+ and CD4+FR-Bh T cells (but not CONT-ENG T cells) were highly reactive to FRα+OC specimens, leading to robust upregulation of multiple activation markers including CD25, CD69, CD137, and PD-1 (FIG. 2A, 2B Upper Panel & FIG. 9B). In contrast, upregulation of these activation markers by FR-Bh T cells was limited/variable in co-cultures with FRα^{lo} specimens (FIG. 2B Lower Panel & FIG. 9B) and were nearly absent when FR-Bh T cells were cultured alone (FIG. 9C), demonstrating FRα-dependent FR-Bh T cell activation. Activation of UTD bystander CD8+ and CD4+ T cells was also observed in FRα+OC patient samples after FR-Bh T cell addition, although the effects were modest compared to transduced FR-Bh T cells (FIG. 9D). Notably, activation of endogenous OC patient CD8+ TALs was readily observed following the addition of FR-Bh T cells but not CONT-ENG T cells to FRα+OC patient samples, with significant upregulation of surface CD25 and CD137 (FIGS. 2C & D), demonstrating effective activation and redirection of endogenous T cells present in the OC TME of human cancer by FR-Bh T cells. Activation of OC patient CD4+ TALs by addition of FR-Bh T cells was also observed (FIG. 9E). These findings indicate that the antitumor response driven by FR-Bh T cells involves both activation of BiTE-producing FR-Bh T cells and engagement of endogenous OC patient T cells present in the peritoneal TME.

### Example 4

### Therapeutic Delivery of BiTE-Secreting T Cells Improves Tumor Control and Survival in an Immunocompetent OC Model

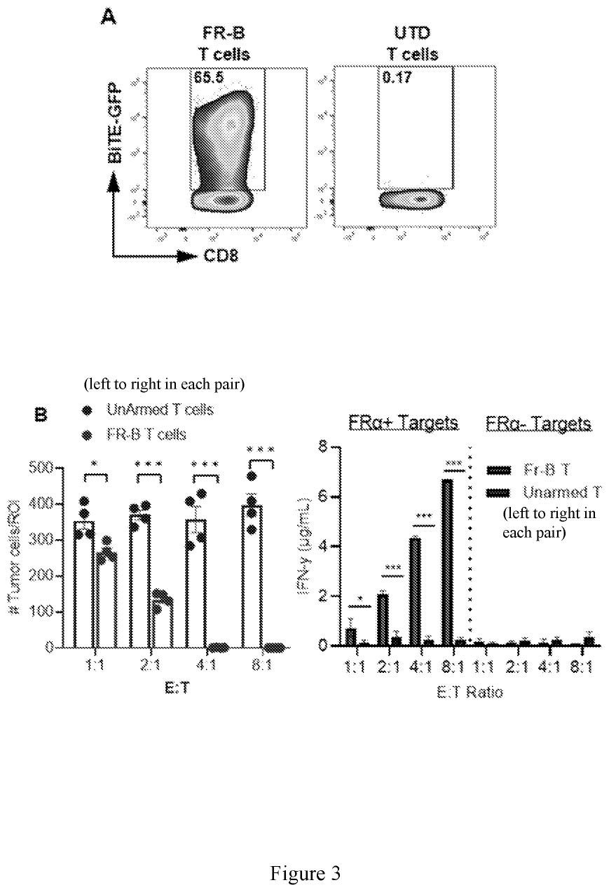

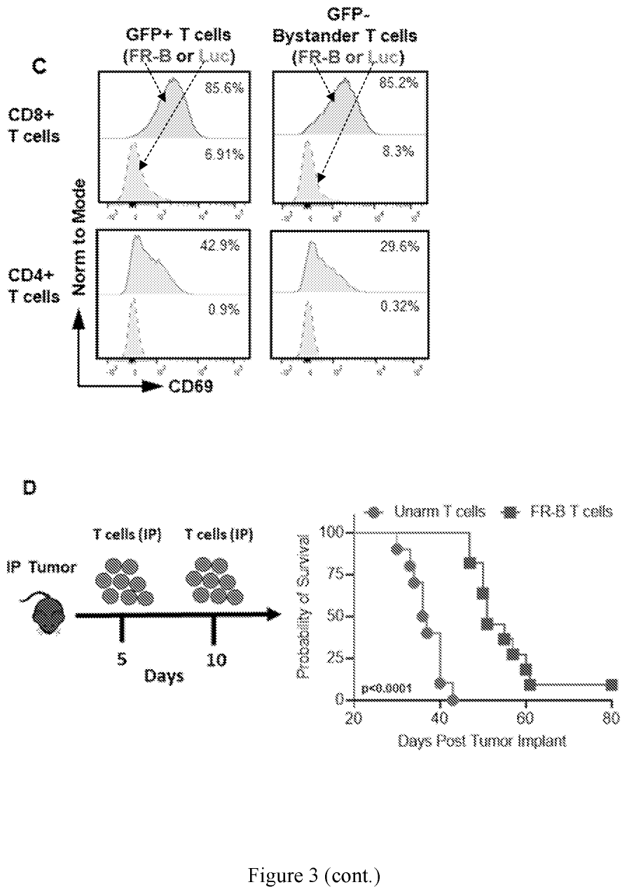

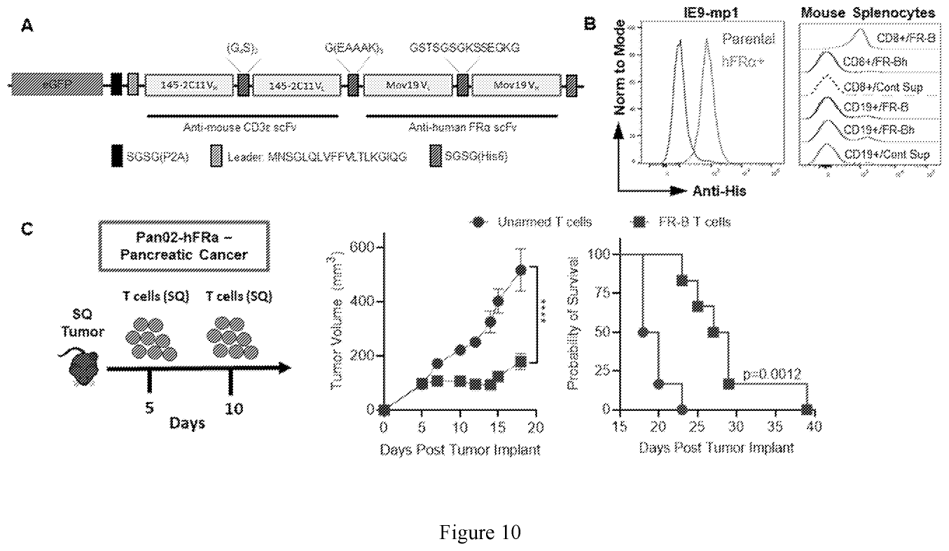

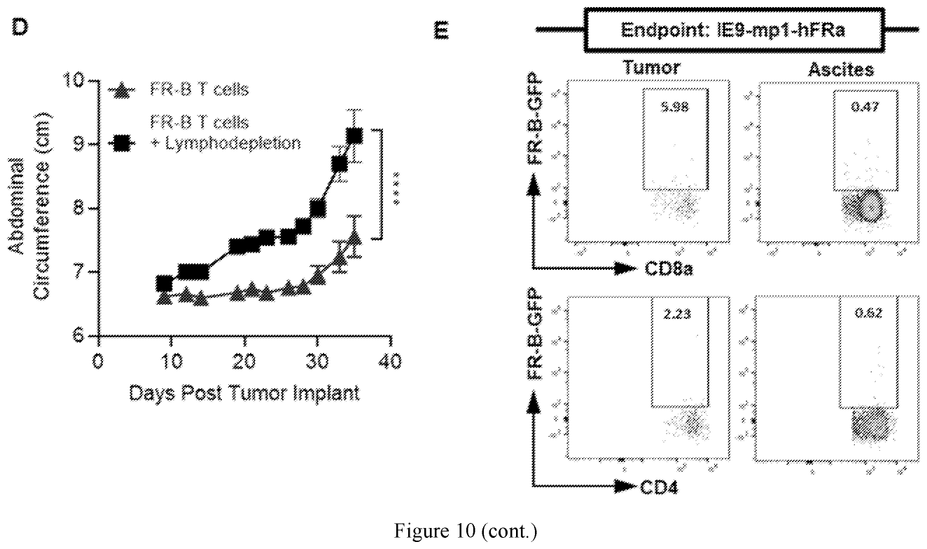

Given that FR-Bh T cells engaged/activated endogenous T cells in OC patient samples, we next tested the therapeutic delivery of FRα-directed BiTE-T cells in an immunocompetent OC mouse model. To do so, an aggressively growing and immunotherapy-resistant variant of the IE9-mp1 OC cell line²⁹ was engineered to stably express human FRα (IE9-mp1-hFRα) and a chimeric BiTE specific for human FRα and mouse CD3ε was generated (hereafter referred to as FR-B) (FIG. 10A). FR-B was confirmed to bind to both IE9-mp1-hFRa target cells and mouse T cells (FIG. 10B). FR-B-secreting T cells (FR-B T cells) were generated with high efficiency from activated mouse splenocytes by retroviral transduction (FIG. 3A) and demonstrated robust killing and antigen-driven effector function in co-culture assays with IE9-mp1-hFRα, but not FRα−parental IE9-mp1 target cells (FIG. 3B). Like human FR-Bh T cells, transduced CD8+ and CD4+FR-B T cells and accompanying UTD bystander T cells were activated in the presence of hFRα+ target cells (FIG. 3C), consistent with FR-B-mediated redirection of bystander T cells.

To evaluate FR-B T cells therapeutically, IE9-mp1-hFRa tumor-bearing mice were treated with FR-B or unarmed control T cells (either UTD or T cells engineered to express a Luciferase-GFP fusion protein; Luc/GFP) and monitored for tumor progression and survival (FIG. 3D). As localized delivery of adoptively transferred CAR-T cells directly into the peritoneal OC TME can effectively control OC progression³⁰⁻³², tumor-bearing mice were treated by IP injection of T cells. Loco-regional delivery of FR-B T cells significantly delayed OC progression compared to control T cells (FIG. 3D, Median survival for Unarmed Control T cells of 36.5 Days compared to 51 days for FR-B T cells) and this effect was confirmed in the subcutaneous Pan02-hFRa tumor model (FIG. 10C). Consistent with data from OC patients demonstrating endogenous T cell activity in response to secreted BiTEs, lymphodepletion prior to tumor implantation/infusion of FR-B T cells led to early tumor progression (FIG. 10D), confirming endogenous T cells are required for optimal tumor control. Analysis of TILs (solid tumor) and TALs (ascites) from IE9-mp1-hFRa tumor-bearing mice at experimental endpoint (due to progressive disease) revealed limited persistence of FR-B T cells (10E), suggesting that tumor outgrowth was associated with clearance of FR-B T cells.

### Example 5

### Stem-Like FR-B T Cells can be Produced Through Cytokine Preconditioning and Improve Antitumor Immunity Following ACT

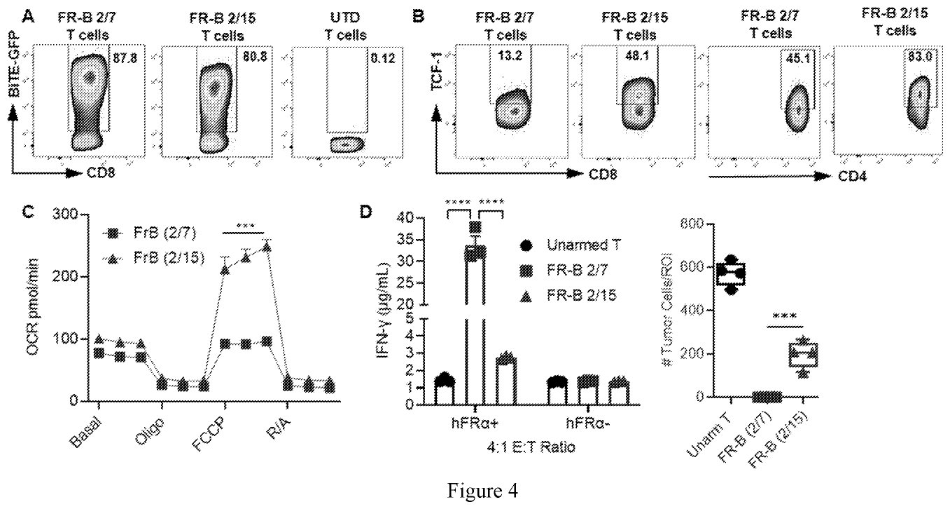

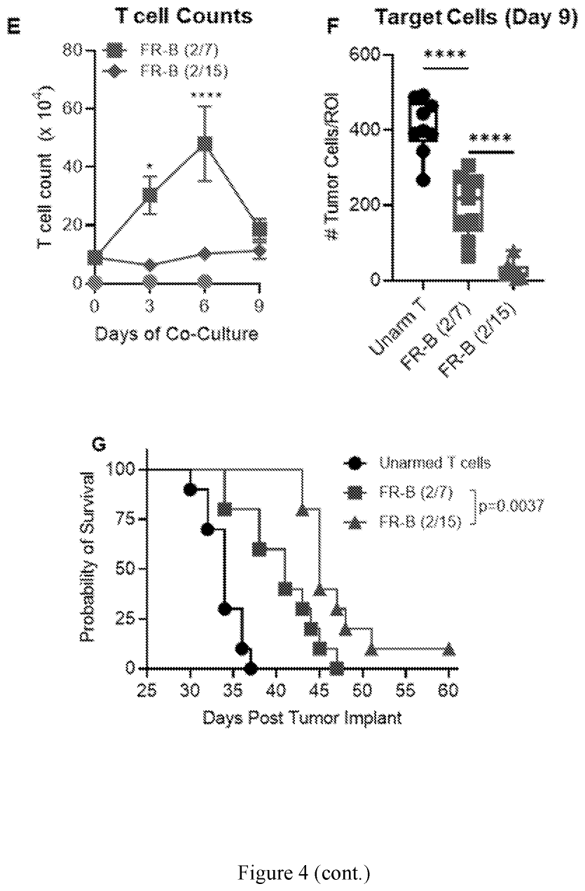

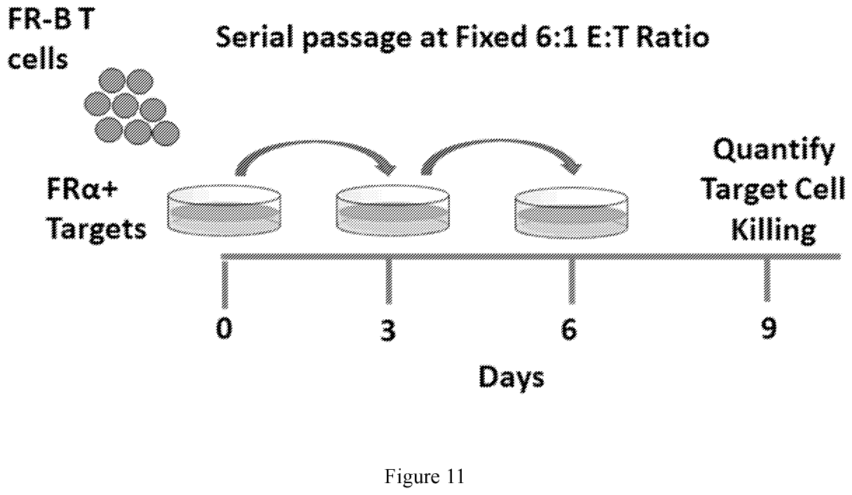

Based on our in vivo findings, we evaluated whether strategies to improve FR-B T cell persistence following infusion would improve therapeutic efficacy. As IL-15 stimulation has been shown to promote a less-differentiated stem cell memory (T_{SCM}) phenotype, increase mitochondrial metabolic fitness, and improve T cell persistence following infusion of CAR-T cells³³, and can enhance the activity of BiTE-T cells³⁴, we tested whether IL-15 preconditioning prior to ACT would impact FR-B T cell efficacy and response durability against OC. As FR-B T cells were produced in the presence of IL-2 and IL-7 (FR-B 2/7) in prior experiments, we directly compared this approach to FR-B T cells produced using IL-2 and IL-15 stimulation (FR-B 2/15). FR-B 2/7 and FR-B 2/15 T cells were generated with similar efficiency by retroviral transduction (FIG. 4A), with FR-B 2/15 T cells having increased TCF-1 expression (FIG. 4B) and an elevated usage of mitochondrial metabolism (FIG. 4C) compared to FR-B 2/7 T cells, consistent with previous data 33. FR-B 2/15 T cells produced more than 10-fold less IFN-γ than FR-B 2/7 T cells (FIG. 4D, Left Panel) and had a reduced capacity to kill IE9-mp1-hFRa cells in co-culture assays (FIG. 4D, Right Panel), consistent with a less differentiated T cell phenotype. However, when tested in an in vitro serial co-culture ‘stress test’ of chronic antigen exposure (FIG. 11), the capacity of FR-B 2/15 T cells to promote durable antitumor activity emerged. While FR-B 2/7 T cells dramatically expanded (>5-fold) prior to abrupt contraction, FR-B 2/15 T cells demonstrated limited expansion in response to antigen stimulation over the entire co-culture period (FIG. 4E). However, while both FR-B 2/7 and FR-B 2/15 T cells cleared all tumor cells in the first two serial co-cultures, FR-B 2/7 T cells developed a reduced ability to lyse IE9-mp1-hFRa tumor cell targets by the third co-culture, while FR-B 2/15 T cell lytic function was maintained (FIG. 4F), suggesting that FR-B 2/15 T cells have a greater capacity to sustain antitumor activity over a prolonged period. When evaluated therapeutically, adoptive transfer of a single dose of FR-B 2/15 T cells 5 days post tumor implantation significantly improved tumor control and long-term survival of IE9-mp1-hFRa tumor-bearing mice compared to FR-B 2/7 T cells (FIG. 4G). Together, these data suggest that generation of FR-B T cells that effectively persist following chronic tumor antigen stimulation led to improved therapeutic efficacy compared to engineered T cells with a heightened capacity for short-term effector function.

### Example 6

### IL-2/IL-15 Preconditioning Improves FR-B T Cell Persistence in the Extratumoral OC Peritoneal TME

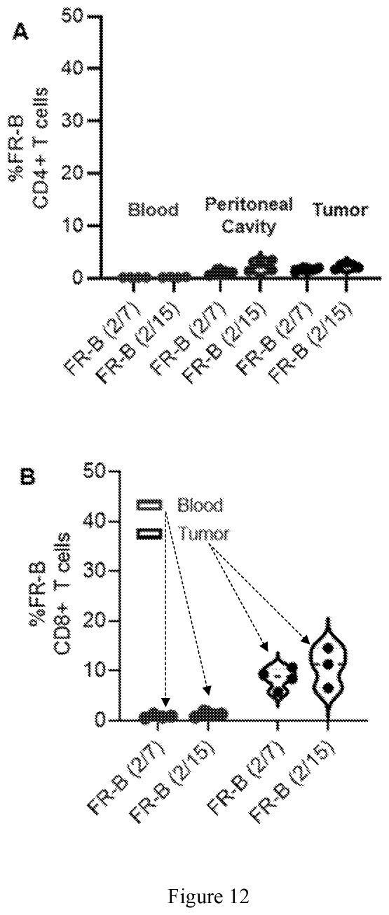

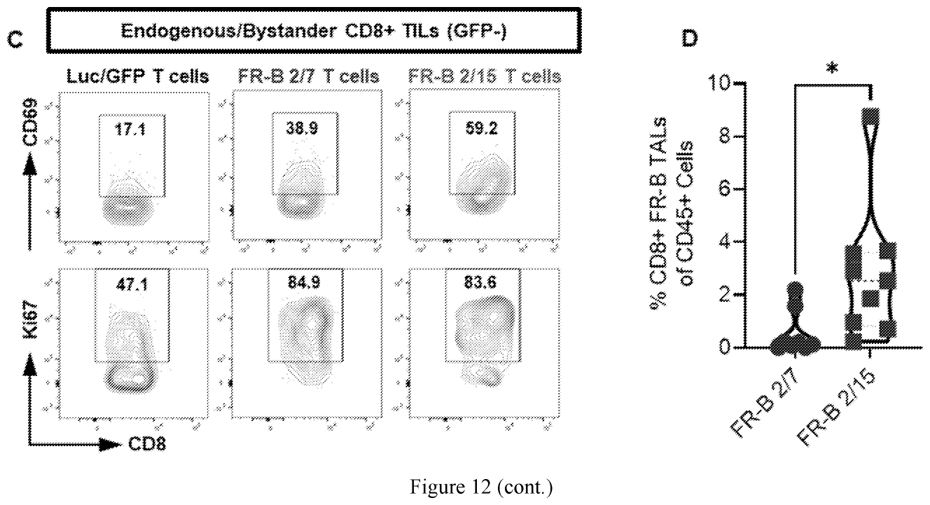

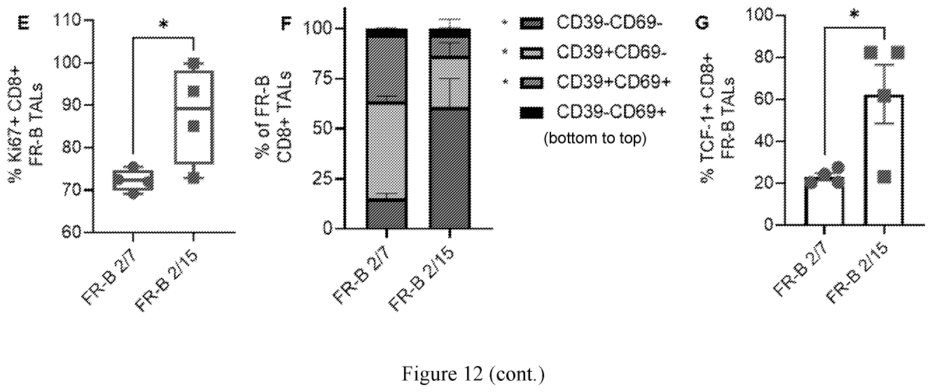

To better understand the improved antitumor effects of FR-B 2/15 T cells, we compared the tissue localization of FR-B 2/7 and FR-B 2/15 T cells following infusion. FR-B CD4+ T cells demonstrated limited accumulation in the blood, peritoneal TME (TALs), as well as solid tumor lesions (TILs), with no clear differences between 2/7 and 2/15 preconditioned FR-B CD4+ T cells (FIG. 12A). FR-B CD8+ T cells had limited accumulation in the blood, with a modest increase in abundance in solid tumor lesions (FIG. 5A and FIG. 12B), consistent with antigen-driven FR-B T cell accumulation at tumor sites. However, there was no difference in the accumulation of FR-B CD8+ TILs between 2/7 and 2/15 conditioned T cells, with the majority of CD8+ TILs (˜90%) comprised of GFP− endogenous and/or bystander T cells (FIG. 5A). Further analysis revealed increased activation (CD69+) and proliferation (Ki67+) of endogenous/bystander CD8+ TILs in response to either FR-B 2/7 or FR-B 2/15 T cell delivery (12C), consistent with data from human OC patients and supporting a mechanistic role for these T cells in the antitumor response.

In contrast to the blood and solid OC tumors, the frequency of FR-B 2/15 CD8+ TALs in the peritoneal cavity was elevated more than 3-fold compared to FR-B 2/7 TALs (FIG. 5B) and comprised an increased proportion of the total CD45+ immune infiltrate in the peritoneal TME (FIG. 12D), suggesting an overall improved capacity of FR-B 2/15 CD8+ T cells to persist in the extratumoral peritoneal OC TME. Moreover, increased Ki67+FR-B 2/15 CD8+ TALs were observed compared to FR-B 2/7 CD8+ TALs (FIG. 5C & FIG. 12E), suggesting ongoing T cell proliferation. Further phenotypic analysis revealed increased accumulation of stem-like CD39-CD69−CD8+ T cells 35 among the FR-B 2/15 CD8+ TALs versus more differentiated CD39+CD69+ T cells present in the FR-B 2/7 CD8+ TALs (FIG. 5D & FIG. 12F). Consistent with this finding, FR-B 2/15 CD8+ TALs also maintained elevated TCF-1 following ACT when compared to the FR-B 2/7 CD8+ TALs (FIG. 5E & FIG. 12G).

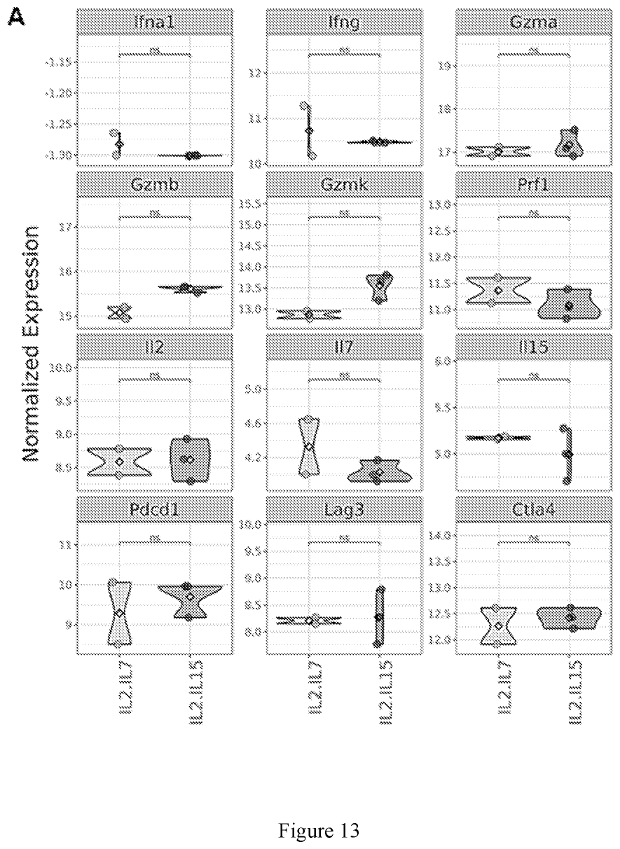

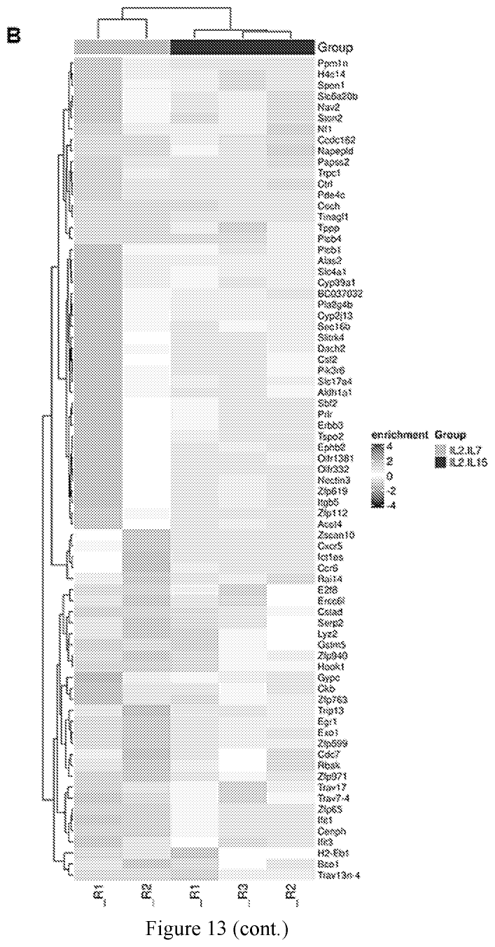

Transcriptional profiling of flow cytometry-sorted CD8+FR-B 2/15 and FR-B 2/7 TALs isolated 5 days post ACT suggested limited differences in effector function or expression of checkpoint pathways between the transferred T cells (FIG. 13A). However, hierarchical clustering of differentially expressed genes revealed FR-B 2/15 and FR-B 2/7 CD8+ TALs to have highly distinct transcriptional profiles (FIG. 13B), with differences in genes associated with multiple cellular processes (FIG. 5F). CD8+FR-B 2/15 TALs had upregulated expression of genes associated with cell proliferation (E2f8, Ercc61, Cenph, Cdc7, Trip13) and cell survival (Ifit3, Egr1), consistent with improved in vivo persistence observed at the cellular level. Additional upregulated genes associated with T cell activation and interferon response (Cstad, Ifit1), as well as cellular metabolism and energy homeostasis (Gstm5, Bco1, Ckb) were observed, suggesting that CD8+FR-B 2/15 TALs can persist as activated T cells, potentially through changes in cellular metabolism. In contrast, CD8+FR-B 2/7 TALs upregulated genes related to apoptotic signaling (Rai14) and negative regulation of transcription and NF-κB signaling (Zscan10, Ppm1n), consistent with poor in vivo persistence and limited T cell activity. Additionally, FR-B 2/7 TALs upregulated genes associated with fatty acid metabolism (Acot4) and regulation of endocytic process (Ston2), increased inflammatory response (CSF2), collagen binding (Coch), extracellular matrix adhesion (Tinagll), as well as responses to extracellular signaling (Pde4c, Plcb4), consistent with interactions between T cells and tumor stroma. Further, upregulation of CXCR5 and CCR6 by CD8+FR-B 2/7 TALs suggested an increased capacity for tissue homing by FR-B 2/7 TALs. Pathway analysis revealed key differences between preconditioning strategies, with FR-B 2/15 TALs enriched for pathways associated with cell replication and T cell function, whereas FR-B 2/7 TALs were enriched for TGF-β responsiveness, chemokine signaling, and ECM interaction (FIG. 5G). Collectively, these data indicate that IL2/IL-15 preconditioned CD8+FR-B TALs have improved persistence and an increased capacity to maintain a less differentiated phenotype, while upregulating cellular processes that serve to maintain FR-B T cell proliferation, survival, and BiTE-driven tumor attack from within the peritoneal OC TME.

References cited for description above follow. This reference listing and the supplemental reference listing not indications that any reference is material to patentability.
1. Rafiq S, Hackett C S, Brentjens R J. Engineering strategies to overcome the current roadblocks in CAR T cell therapy. Nat Rev Clin Oncol 2020; 17(3):147-67. doi: 10.1038/s41571-019-0297-y [published Online First: 2019/12/19]
2. Rosenberg S A, Yang J C, Sherry R M, et al. Durable complete responses in heavily pretreated patients with metastatic melanoma using T-cell transfer immunotherapy. Clin Cancer Res 2011; 17(13):4550-7. doi: 10.1158/1078-0432.Ccr-11-0116 [published Online First: 2011/04/19]
3. Creelan B C, Wang C, Teer J K, et al. Tumor-infiltrating lymphocyte treatment for anti-P D-1-resistant metastatic lung cancer: a phase 1 trial. Nat Med 2021; 27(8):1410-18. doi: 10.1038/s41591-021-01462-y [published Online First: 2021/08/14]
4. Kverneland A H, Chamberlain C A, Borch T H, et al. Adoptive cell therapy with tumor-infiltrating lymphocytes supported by checkpoint inhibition across multiple solid cancer types. J Immunother Cancer 2021; 9(10) doi: 10.1136/jitc-2021-003499 [published Online First: 2021/10/06]
5. Kandalaft L E, Dangaj Laniti D, Coukos G. Immunobiology of high-grade serous ovarian cancer: lessons for clinical translation. Nat Rev Cancer 2022; 22(11):640-56. doi: 10.1038/s41568-022-00503-z [published Online First: 2022/09/16]
6. Chodon T, Lugade A A, Battaglia S, et al. Emerging Role and Future Directions of Immunotherapy in Advanced Ovarian Cancer. Hematol Oncol Clin North Am 2018; 32(6):1025-39. doi: 10.1016/j.hoc.2018.07.011 [published Online First: 2018/11/06]
7. Yang C, Xia B R, Zhang Z C, et al. Immunotherapy for Ovarian Cancer: Adjuvant, Combination, and Neoadjuvant. Front Immunol 2020; 11:577869. doi: 10.3389/fimmu.2020.577869 [published Online First: 2020/10/31]
8. Sato E, Olson S H, Ahn J, et al. Intraepithelial CD8+ tumor-infiltrating lymphocytes and a high CD8+/regulatory T cell ratio are associated with favorable prognosis in ovarian cancer. Proc Natl Acad Sci USA 2005; 102(51):18538-43. doi: 10.1073/pnas.0509182102 [published Online First: 2005/12/14]
9. Scheper W, Kelderman S, Fanchi L F, et al. Low and variable tumor reactivity of the intratumoral TCR repertoire in human cancers. Nat Med 2019; 25(1):89-94. doi: 10.1038/s41591-018-0266-5 [published Online First: 2018/12/05]
10. Simoni Y, Becht E, Fehlings M, et al. Bystander CD8(+) T cells are abundant and phenotypically distinct in human tumour infiltrates. Nature 2018; 557(7706):575-79. doi: 10.1038/s41586-018-0130-2 [published Online First: 2018/05/18]
11. Scott A C, Dundar F, Zumbo P, et al. TOX is a critical regulator of tumour-specific T cell differentiation. Nature 2019; 571(7764):270-74. doi: 10.1038/s41586-019-1324-y [published Online First: 2019/06/18]
12. Slaney C Y, Wang P, Darcy P K, et al. CARs versus BiTEs: A Comparison between T Cell-Redirection Strategies for Cancer Treatment. Cancer Discov 2018; 8(8):924-34. doi: 10.1158/2159-8290.Cd-18-0297 [published Online First: 2018/07/18]
13. Wang Q, Ma X, Wu H, et al. Oncolytic adenovirus with MUC16-BiTE shows enhanced antitumor immune response by reversing the tumor microenvironment in PDX model of ovarian cancer. Oncoimmunology 2022; 11(1):2096362. doi: 10.1080/2162402x.2022.2096362 [published Online First: 2022/07/09]
14. Yeku 00, Rao T D, Laster I, et al. Bispecific T-Cell Engaging Antibodies Against MUC16 Demonstrate Efficacy Against Ovarian Cancer in Monotherapy and in Combination With PD-1 and VEGF Inhibition. Front Immunol 2021; 12:663379. doi: 10.3389/fimmu.2021.663379 [published Online First: 2021/05/04]
15. Avanzino B C, Prabhakar K, Dalvi P, et al. A T-cell engaging bispecific antibody with a tumor-selective bivalent folate receptor alpha binding arm for the treatment of ovarian cancer. Oncoimmunology 2022; 11(1):2113697. doi: 10.1080/2162402x.2022.2113697 [published Online First: 2022/08/27]
16. Zhu M, Wu B, Brandl C, et al. Blinatumomab, a Bispecific T-cell Engager (BiTE(®)) for CD-19 Targeted Cancer Immunotherapy: Clinical Pharmacology and Its Implications. Clin Pharmacokinet 2016; 55(10):1271-88. doi: 10.1007/s40262-016-0405-4 [published Online First: 2016/05/23]
17. Belmontes B, Sawant D V, Zhong W, et al. Immunotherapy combinations overcome resistance to bispecific T cell engager treatment in T cell-cold solid tumors. Sci Transl Med 2021; 13(608) doi: 10.1126/scitranslmed.abd1524 [published Online First: 2021/08/27]
18. Iwahori K, Kakarla S, Velasquez M P, et al. Engager T cells: a new class of antigen-specific T cells that redirect bystander T cells. Mol Ther 2015; 23(1):171-8. doi: 10.1038/mt.2014.156 [published Online First: 2014/08/22]
19. Bonifant C L, Szoor A, Torres D, et al. CD123-Engager T Cells as a Novel Immunotherapeutic for Acute Myeloid Leukemia. Mol Ther 2016; 24(9):1615-26. doi: 10.1038/mt.2016.116 [published Online First: 2016/07/13]
20. Li G, Reid K M, Spitler K, et al. CD3 engagement as a new strategy for allogeneic “off-the-shelf” T cell therapy. Mol Ther Oncolytics 2022; 24:887-96. doi: 10.1016/j.omto.2022.02.024 [published Online First: 2022/03/24]
21. Choi B D, Yu X, Castano A P, et al. CAR-T cells secreting BiTEs circumvent antigen escape without detectable toxicity. Nat Biotechnol 2019; 37(9):1049-58. doi: 10.1038/s41587-019-0192-1 [published Online First: 2019/07/25]
22. Vergote I B, Marth C, Coleman R L. Role of the folate receptor in ovarian cancer treatment: evidence, mechanism, and clinical implications. Cancer Metastasis Rev 2015; 34(1):41-52. doi: 10.1007/s10555-014-9539-8 [published Online First: 2015/01/08]
23. Scaranti M, Cojocaru E, Banerjee S, et al. Exploiting the folate receptor α in oncology. NatRev Clin Oncol 2020; 17(6):349-59. doi: 10.1038/s41571-020-0339-5 [published Online First: 2020/03/11]
24. Chen Y L, Chang M C, Huang C Y, et al. Serous ovarian carcinoma patients with high alpha-folate receptor had reducing survival and cytotoxic chemo-response. Mol Oncol 2012; 6(3):360-9. doi: 10.1016/j.molonc.2011.11.010 [published Online First: 2012/01/24]
25. Zamarin D, Walderich S, Holland A, et al. Safety, immunogenicity, and clinical efficacy of durvalumab in combination with folate receptor alpha vaccine TPIV200 in patients with advanced ovarian cancer: a phase II trial. J Immunother Cancer 2020; 8(1) doi: 10.1136/jitc-2020-000829 [published Online First: 2020/06/07]
26. Moore K N, Oza A M, Colombo N, et al. Phase III, randomized trial of mirvetuximab soravtansine versus chemotherapy in patients with platinum-resistant ovarian cancer: primary analysis of FORWARD I. Ann Oncol 2021; 32(6):757-65. doi: 10.1016/j.annonc.2021.02.017 [published Online First: 2021/03/06]
27. Shah P, Shlanksy-Goldberg R, Martin L, et al. 431 First-in-human phase I clinical trial evaluating intraperitoneal administration of MOv19-BBz CAR T cells in patients with alpha folate receptor-expressing recurrent high grade serous ovarian cancer. Journal for ImmunoTherapy of Cancer 2021; 9(Suppl 2):A461-A61. doi: 10.1136/jitc-2021-SITC2021.431
28. FDA Gives Nod to Mirvetuximab Soravtansine. Cancer Discov 2022:Ofl. doi: 10.1158/2159-8290.Cd-nb2022-0075 [published Online First: 2022/11/29]
29. McGray A J R, Huang R Y, Battaglia S, et al. Oncolytic Maraba virus armed with tumor antigen boosts vaccine priming and reveals diverse therapeutic response patterns when combined with checkpoint blockade in ovarian cancer. J Immunother Cancer 2019; 7(1):189. doi: 10.1186/s40425-019-0641-x [published Online First: 2019/07/19]
30. Yeku O O, Purdon T J, Koneru M, et al. Armored CAR T cells enhance antitumor efficacy and overcome the tumor microenvironment. Sci Rep 2017; 7(1):10541. doi: 10.1038/s41598-017-10940-8 [published Online First: 2017/09/07]
31. Koneru M, Purdon T J, Spriggs D, et al. IL-12 secreting tumor-targeted chimeric antigen receptor T cells eradicate ovarian tumors in vivo. Oncoimmunology 2015; 4(3):e994446. doi: 10.4161/2162402x.2014.994446 [published Online First: 2015/05/08]
32. Murad J P, Kozlowska A K, Lee H J, et al. Effective Targeting of TAG72(+) Peritoneal Ovarian Tumors via Regional Delivery of CAR-Engineered T Cells. Front Immunol 2018; 9:2268. doi: 10.3389/fimmu.2018.02268 [published Online First: 2018/12/05]
33. Alizadeh D, Wong R A, Yang X, et al. IL15 Enhances CAR-T Cell Antitumor Activity by Reducing mTORC1 Activity and Preserving Their Stem Cell Memory Phenotype. Cancer Immunol Res 2019; 7(5):759-72. doi: 10.1158/2326-6066.Cir-18-0466 [published Online First: 2019/03/21]
34. Mu-Mosley H, Ostermann L, Muftuoglu M, et al. Transgenic Expression of IL15 Retains CD123-Redirected T Cells in a Less Differentiated State Resulting in Improved Anti-AML Activity in Autologous AML PDX Models. Front Immunol 2022; 13:880108. doi: 10.3389/fimmu.2022.880108 [published Online First: 2022/05/27]
35. Krishna S, Lowery F J, Copeland A R, et al. Stem-like CD8 T cells mediate response of adoptive cell immunotherapy against human cancer. Science 2020; 370(6522):1328-34. doi: 10.1126/science.abb9847 [published Online First: 2020/12/12]
36. Zangari B, Tsuji T, Matsuzaki J, et al. Tcf-1 protects anti-tumor TCR-engineered CD8(+) T-cells from GzmB mediated self-destruction. Cancer Immunol Immunother 2022; 71(12):2881-98. doi: 10.1007/s00262-022-03197-2 [published Online First: 2022/04/24]
37. Li Z, Tuong Z K, Dean I, et al. In vivo labeling reveals continuous trafficking of TCF-1+ T cells between tumor and lymphoid tissue. J Exp Med 2022; 219(6) doi: 10.1084/jem.20210749 [published Online First: 2022/04/27]
38. Patsoukis N, Bardhan K, Chatterjee P, et al. PD-1 alters T-cell metabolic reprogramming by inhibiting glycolysis and promoting lipolysis and fatty acid oxidation. Nat Commun 2015; 6:6692. doi: 10.1038/ncomms7692 [published Online First: 2015/03/27]
39. Vignali PDA, DePeaux K, Watson M J, et al. Hypoxia drives CD39-dependent suppressor function in exhausted T cells to limit antitumor immunity. Nat Immunol 2022 doi: 10.1038/s41590-022-01379-9 [published Online First: 2022/12/22]
40. Tay R E, Richardson E K, Toh H C. Revisiting the role of CD4(+) T cells in cancer immunotherapy-new insights into old paradigms. Cancer Gene Ther 2021; 28(1-2):5-17. doi: 10.1038/s41417-020-0183-x [published Online First: 2020/05/28]
41. Andreatta M, Corria-Osorio J, Müller S, et al. Interpretation of T cell states from single-cell transcriptomics data using reference atlases. Nat Commun 2021; 12(1):2965. doi: 10.1038/s41467-021-23324-4 [published Online First: 2021/05/22]
42. Narayan V, Barber-Rotenberg J S, Jung I Y, et al. PSMA-targeting TGFβ-insensitive armored CAR T cells in metastatic castration-resistant prostate cancer: a phase 1 trial. Nat Med 2022; 28(4):724-34. doi: 10.1038/s41591-022-01726-1 [published Online First: 2022/03/23]
43. Blanco B, Ramirez-Fernández Á, Bueno C, et al. Overcoming CAR-Mediated CD19 Downmodulation and Leukemia Relapse with T Lymphocytes Secreting Anti-CD19 T-cell Engagers. Cancer Immunol Res 2022; 10(4):498-511. doi: 10.1158/2326-6066.Cir-21-0853 [published Online First: 2022/04/02]
44. Cao G, Zhang G, Liu M, et al. GPC3-targeted CAR-T cells secreting B7H3-targeted BiTE exhibit potent cytotoxicity activity against hepatocellular carcinoma cell in the in vitro assay. Biochem Biophys Rep 2022; 31:101324. doi: 10.1016/j.bbrep.2022.101324 [published Online First: 2022/08/30]
45. Yin Y, Rodriguez J L, Li N, et al. Locally secreted BiTEs complement CAR T cells by enhancing killing of antigen heterogeneous solid tumors. Mol Ther 2022; 30(7):2537-53. doi: 10.1016/j.ymthe.2022.05.011 [published Online First: 2022/05/17]
46. Liang Z, Dong J, Yang N, et al. Tandem CAR-T cells targeting FOLR1 and MSLN enhance the antitumor effects in ovarian cancer. Int J Biol Sci 2021; 17(15):4365-76. doi: 10.7150/ijbs.63181 [published Online First: 2021/11/23]

### Discussion of Examples Above

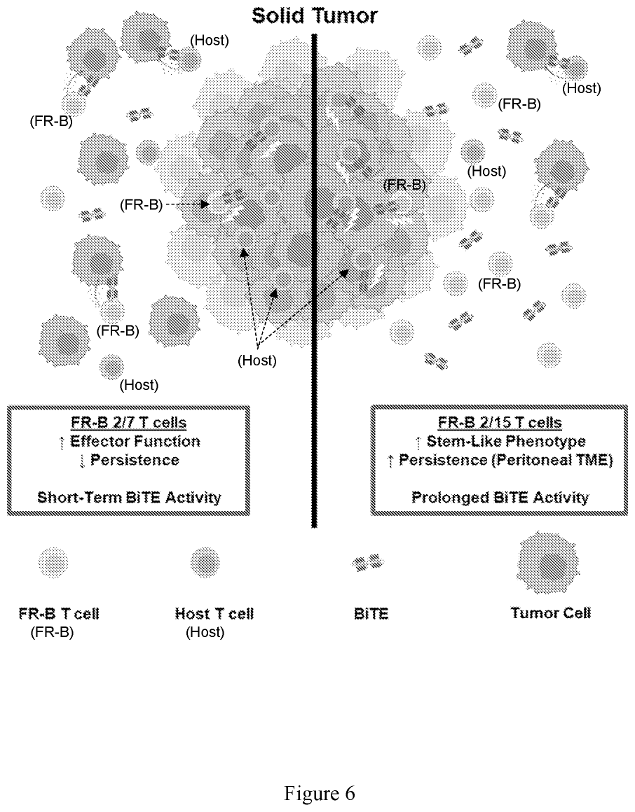

The present disclosure demonstrates robust activity of FR-B(h) T cells that was accompanied by engagement of endogenous T cells in the OC TME, thereby overcoming limited endogenous immunoreactivity or local tumor immunosuppression. Delivery of T cells by IP injection has been shown to result in accumulation of infused T cells in solid tumors in the peritoneal cavity³², which is consistent with the present data shown for FR-B T cells. However, FR-B T cells comprised only a small fraction of the TILs found in solid OC and the improved therapeutic effects of FR-B 2/15 over FR-B 2/7 T cell therapy correlated with differences in FR-B T cell accumulation outside of solid tumors (FIG. 6). Unlike conventional ACT approaches (Eg. engineering tumor reactive CAR- or TCR-T cells) where therapeutic failure can stem from limited tumor infiltration following T cell infusion, the capacity of the described BiTE-T cells to mediate tumor attack from remote location(s) outside of solid tumors provides an approach for generating durable responses that differs from previous approaches.

A recent report demonstrated that tumor-specific CD8+ T cells that infiltrate and remain in tumors for at least 72 hrs upregulate checkpoint receptors and can rapidly develop an exhausted phenotype³⁷, emphasizing that i) limiting BiTE-T cell infiltration into tumors may be beneficial for prolonging BiTE-T cell activity and ii) the activation of endogenous T cells by secreted BiTEs indicates the presence of newly infiltrating (and not yet exhausted) tumor-specific T cells or activation of bystander T cells that remain functional in the TME. Additionally, the instant data indicate that effector-like FR-B 2/7 CD8+ TALs increase fatty acid/lipid metabolism within the OC TME, metabolic reprogramming that has been associated with PD-1 signaling³⁸ and suggesting FR-B T cells can also be impacted by inhibitory cues in the broader peritoneal OC TME that may promote early T cell clearance. Moreover, a recent report demonstrated that CD39-expressing CD8+ T cells can directly suppress the antitumor activity of tumor-specific T cells³⁹ suggests the predominantly CD39+FR-B 2/7 FR-B TALs may actually limit tumor attack within the OC TME.

The disclosure includes use of the FR-B T cells to localize to other sites in the peritoneal space, including tumor-draining lymph nodes or the spleen. A small frequency of FR-B T cells was also observed in circulation, supporting loco-regional delivery of FR-B T cells leading to antitumor immunity at distant metastatic sites.

In addition to elevated IFN-γ levels, we also noted increased production of Th2-associated cytokines (in particular IL-5) in response to FR-Bh T cells in OC patient samples, suggesting BiTE-driven activity of diverse T cell subsets. Engaging multiple T cell subsets, whether CD8+ T cells or differentially polarized CD4+ T cells (which can include Tregs)⁴⁰ may impact therapeutic response, particularly as T cells present in the TME at the time of ACT may exist in multiple heterogenous states⁴¹. We also observed a reduction in IL-6 in OC patient samples treated with FR-Bh T cells, which contrasts with CAR-T cell therapy where elevated IL-6 has been associated with increasing severity of cytokine release syndrome⁴².

The disclosure includes use of the described BiTE-T cells with a co-stimulatory signal and/or cytokines. It is considered that because soluble BiTEs effectively combine with blockade of checkpoint receptors including PD-1 and CTLA-4¹⁷, it is considered likely that the described FR-B(h) T cells will synergize with checkpoint blockade for treating OC.

The disclosure includes multi-arming T cells, for example with CARs and the described BiTEs to target multiple tumor antigens which may overcome tumor heterogeneity and/or elicit immune attack on multiple target cell subsets.

The presently provided results demonstrate the potent effects of FR-B(h) T cells for ACT in OC, which can effectively redirect endogenous T cells to amplify antitumor immunity. The results also reveal a unique attribute of FR-B T cells in OC to persist and direct antitumor activity from solid tumor-adjacent or extratumoral locations in the peritoneal TME, which may have distinct mechanistic advantages for enhancing response durability following ACT.

### Example 7

### This Example Provides Supplemental Methods in Respect of the Prior Examples.

Generation of hFra-Expressing Cell Lines, BiTE Constructs and Retroviral Constructs

Human FRα (hFRa) was PCR amplified from cDNA of SK-MEL-37 melanoma cell line using the following primers

FWD:
(SEQ ID NO: 11)
TGTCGTGAAAACTACCCCGCGGCCGCCACCATGGCTCAGCGGATGACAAC
ACA
and
REV:
(SEQ ID NO: 12)
TTCGTGGCTCCGGAGCCACTGCTGAGCAGCCACAGCAGCATT.

The hFRa gene was genetically fused to the monomeric enhanced GFP (eGFP) reporter via SGSG-linker and a P2A translational skipping sequence and inserted into the pT2-EF 30 sleeping beauty transposon plasmid¹ using NEBuilder® HIFI DNA assembly (New England Biolabs), with sequences confirmed by Sanger Sequencing at the Roswell Park Genomics Shared Resource. The pT2-EF-hFRα-GFP vector was co-electroporated with the CMV(CAT)T7-SB100 transposase vector (Addgene plasmid #34879; RRID:Addgene_34879) into IE9-mp1 and Pan02 cell lines using the Nucleofector 4D Instrument. Electroporated cells were cultured for 10-14 days prior to FACs sorting of GFP^{hi} cells using a BD FACSAria II cell sorter. Sorted cells were confirmed to express hFRa by flow cytometry. A hFRα-specific scFv with murine immunoglobulin kappa light chain was designed by fusing MOv19 kappa chain (Sequence ID: X99994.1) and heavy chain (Sequence ID: X99993.1) sequences via a 212 polypeptide-containing linker (GSTSGSGKSSEGKG (SEQ ID NO:13)) and was synthesized by Integrated DNA technologies gBlock. FR—B is a chimeric BiTE that binds human FRα and mouse CD3e (via a previously described scFv derived from the 145-2C11 monoclonal antibody². These scFvs are linked by a rigid and long G(EAAAK SEQ ID NO:9)x3 linker sequence that resulted in optimal antigen binding and in vivo FR-B activity compared to a panel of tested linkers (data not shown). The BiTE leader sequence² and 145-2C11 derived scFv sequence were codon-optimized and synthesized by gBlock (Integrated DNA technologies), with the FR-B sequence designed to contain a 6× His Tag at the C terminus. The FR-B sequence was genetically fused to the monomeric enhanced GFP (eGFP) reporter via a SGSG (SEQ ID NO:9)-linker and P2A translational skipping sequence to allow monitoring of transduction efficiency (FIG. 10A). The FR-Bh sequence was generated from the FR-B BiTE by exchanging the 145-2C11 derived mouse CD3ε binding scFv with the human CD3R-specific UCHT1 scFv sequence ordered from Integrated DNA technologies as a gBlock containing the same BiTE leader sequence as above and the UCHT1 scFv). The DNA sequence corresponding to the G(EAAAK)3 (SEQ ID NO:9) linker (GEAAAKEAAAKEAAAK (SEQ ID NO:8), followed by Mov19 scFv, 6× His Tag, Furin cleavage peptide, SGSG linker+P2A translational skipping sequence, and monomeric enhanced GFP (eGFP) reporter (to monitor transduction efficiency) was PCR amplified from an existing plasmid to generate overlapping DNA fragments amenable to assembly using NEBuilder (FIG. 7A). FR—B and FR-Bh sequences along with GFP reporter genes were inserted into the previously described retroviral vector¹ using NotI and PacI restriction sites, with DNA fragments assembled using NEBuilder HIFI DNA assembly (New England Biolabs). Plasmid sequences were confirmed by Sanger Sequencing, and retroviruses used to transduce human or mouse cells produced in PG13 or PLAT-E retroviral packaging cells lines, respectively. For murine studies, control T cells were either transduced with a retrovirus expressing a codon-optimized Luciferase (Luc2)-P2A-GFP gene and produced in PLAT-E cells or were Mock transduced. For human T cells, Control Engager secreting (Cont-ENG) T cells were generated by transducing human T cells with the FR-B retroviral vector (produced in PG13 cells), where secreted Engagers can bind FRα+ target cells, but not human T cells due to lack of cross-reactivity of the 145-2C11 scFv with human CD3 (confirmed by flow cytometry), thus preventing Engager-mediated T cell activation upon FRα binding. In all cases, cell culture supernatant from high-titer retrovirus producing clones was collected and used for viral transduction. For initial testing of BiTEs for antigen-specific binding, 293T cells were retrovirally transduced, followed by collection of 293T cell culture supernatants. Supernatants were spun at 1500 rpm for 5 mins to remove cells/debris, followed by 20-40× concentration using Corning Spin-X® UF20 30k MWCO columns according to the manufacturers recommended protocol. Concentrated supernatants were added to target cells and incubated for 90 mins at 4° C. with supernatants from unmodified 239T cells used as controls. Following incubation, cells were washed and subsequently stained with an anti-His Tag antibody for detection of BiTE binding to target cells by flow cytometry as described separately above.

### T Cell Activation and Transduction

For human studies, peripheral blood mononuclear cells (PBMC) were isolated from the whole blood of healthy donors and received under an approved Biospecimen and Data Research (BDR) protocol. PMBC were activated using precoated plate-bound anti-human CD3ε antibody (OKT-3, 5 μg/ml prepared in PBS, Bio X Cell) for 48 hrs in cRPMI containing anti-human CD28 antibody (9.3, 2 μg/ml, Bio X Cell), human IL-2 (50 U/ml, Peprotech), and human IL-7 (10 ng/ml, BioLegend). For murine studies, splenocytes were harvested from female C57BL/6J or T-Lux 3 mice, subjected to RBC lysis using ACK lysis buffer, and activated using precoated plate-bound anti-mouse CD3ε (145-2C11, 5 μg/ml prepared in PBS, Bio X cell) for 48-72 hrs in cRPMI containing anti-mouse CD28 antibody (37.51, 2 μg/ml, Bio X Cell), human IL-2 (50 U/ml, Peprotech), and either mouse IL-7 (10 ng/ml, BioLegend) or mouse IL-15 (10 ng/ml BioLegend). Following activation, T cells were harvested, counted, and loaded onto Retronectin (Takara)-coated non-tissue culture treated plates preloaded with retrovirus by spinning cleared cell supernatants from high-titer retrovirus-producing PG13 (human) or PLAT-E (mouse) cells at 3000 rpm for 1 hr at 32° C. (2 cycles of retrovirus preloading completed prior to T cell loading). T cell transduction was conducted on two consecutive days, followed by at least 24 hr T cell expansion prior to assessment of T cell transduction efficiency (based on GFP+ cells, gated using GFP− mock transduced T cells) by flow cytometry. Following activation, T cells were maintained in cRPMI containing cytokine support (IL-2+IL-7 or IL-15), which was replaced every 2-3 days.

### Antibodies and Flow Cytometry Staining/Analysis

For mouse samples, Fc blocking was performed by using an anti-CD16/CD32 antibody (2.4G2, Bio X Cell, 15 min at 4° C.) to inhibit non-specific antibody binding prior to surface staining. For intracellular staining, The BD Transcription Factor Buffer Set (BD Biosciences) was used according to the manufacturer's suggested protocol. In cases where fixation/permeabilization were performed, intracellular staining for GFP was additionally included to permit interrogation of FR-B T cells based on GFP in fixed cells. For human samples, blocking was performed using human FcR Block (Miltenyi Biotech) for 10 min at 4° C., followed by staining (30 mins, 4° C.) using antibodies prepared in either FACs Buffer or BD Horizon™ Brilliant Staining Buffer. For human studies involving addition of engineered T cells to OC patient specimens, CONT-ENG or FR-Bh T cells were harvested and pre-labeled with CellTrace Violet (Thermo Fisher) according to the recommended protocol prior to addition. For all direct ex vivo mouse studies and for studies using human OC specimens, Zombie UV or Zombie Near-IR fixable viability staining (BioLegend) was performed to ensure interrogation of only viable cells. Stained Samples were collected using BD LSR II or Fortessa Flow cytometers and downstream data analyzed using FlowJo V10 software (BD Biosciences).

### In Vitro Co-Culture Studies

To assess target cell killing, T cells were gently washed from cultures using cold PBS and target cells were enumerated by counting a minimum of 4 randomly selected regions of interest (ROI's)/well using the Cytation 5 instrument (Biotek) or quantified using the CellTiter-Glo 2.0 Cell Viability Assay (Promega) to determine % target cell killing compared to control wells containing target cells alone. T cell activation (FR-B, Luc/GFP, or Mock transduced T cells) was assessed after 24 hr co-culture with IE9-mp1-hFRa target cells using CD69 surface staining and flow cytometry. Culture supernatants from co-cultures were collected, spun down to remove debris, aliquoted and stored at −80° C. prior to analysis and were assessed for IFN-γ production using either the human or mouse IFN-γ ELISA MAX Deluxe set (BioLegend) according to the manufacturer's suggested protocol.

### Transwell Assay to Assess BiTE-Mediated Bystander T Cell Activity

2×10⁵ untransduced (UTD) human T cells were plated with (2×10⁴) SK-OV-6 cells (E:T=10:1) in 24 well tissue culture plates in 800 μl cRPMI. Next, 0.4 m PETMembrane 24 well transwell inserts (Greiner Bio-One) were placed in the wells and 10⁶ human CONT-ENG or FR-Bh T cells were added to the transwell in 200 μl cRPMI. CONT-ENG or FR-Bh T cells added directly to the SK-OV-6 target cells (no transwell) were used as negative and positive controls, respectively. Co-cultures were plated in technical duplicate or triplicate and incubated for 48 hrs in the presence of human IL-2 (50 U/ml), at which point T cells were gently washed from the lower chamber and target cells harvested and viable cells counted. Culture supernatants were collected and analyzed for human IFN-γ production by ELISA as described.

### OC Patient Samples and Targeting Using FR-Bh T Cells

Patient samples were assessed for the frequency of tumor cells (CD45-EpCAM+) that were FRα+ by flow cytometry. Following 48 hr co-culture, culture supernatants were collected for downstream analysis and cells collected for flow cytometry as detailed. To allow enumeration of FRα+ and FRα−tumor cells following co-culture, CountBright Absolute Counting Beads (Thermo Fisher) were added prior to Flow cytometry analysis. Culture supernatants were spun down to remove debris, aliquoted and stored at −80° C. prior to analysis and were assessed for IFN-γ production using the human IFN-γ ELISA MAX Deluxe set (BioLegend) according to the manufacturer's suggested protocol. Additionally, 4 responding patient culture supernatants were selected to assess broad immunological effects between OC patient samples containing either CONT-ENG or FR-Bh T cells using the Isoplexis Human Adaptive Immune Codeplex Secretome to measure 22 inflammatory parameters using the Isoplexis Isolight system according to the manufacturer's recommended protocol.

### Preclinical Mouse Models and Therapeutic Delivery of FR-B T Cells

For evaluation of FR-Bh T cell therapeutic activity using a human xenograft model, 3×10⁶ SK-OV-6 cells (prepared in 100 μl PBS) were implanted subcutaneously in the flanks of female 6-8-week-old female NSG mice bred in the Roswell Park Laboratory Animal Shared Resource (LASR). Tumor volumes were calculated as 0.5×(Length×Width²) and when tumors reached ˜150 mm³, mice were stratified into groups to remain untreated or receive 3×10⁶ FR-Bh or Cont-ENG T cells (delivered as split dose between IV and intratumoral routes, prepared in 200 μl PBS). Mice received 3 daily doses of 2×10⁴ U of IL-2 (IP injection, 200 μl PBS) beginning on the day of T cell infusion. Changes in tumor volume were determined twice/week for the duration of study. In studies involving depletion of endogenous lymphocytes in immunocompetent mice, mice were treated with 5 Gy Total body irradiation immediately prior to tumor implantation and lymphodepletion confirmed by flow cytometry prior to adoptive T cell transfer. IP tumor progression was monitored based on increased abdominal distension (measured as changes in circumference) due to accumulation of peritoneal ascites, which closely correlates with solid tumor growth in this model^{4 5}, with mice considered endpoint and euthanized when abdominal circumference reached 10 cm (or at earlier measurements if mice developed decreasing health status due to peritoneal disease progression). For subcutaneously implanted Pan02-hFRα, tumor volume was calculated at 0.5×(Length×Width²), with mice considered endpoint and euthanized when tumor dimensions exceed 10 mm in both directions. All performed experiments and procedures were reviewed and approved by the Roswell Park IACUC prior to conducting experiments.

### Monitoring of FR-B T Cell Responses in Treated Mice

Blood was collected by retro-orbital blood draw, peritoneal lavage collected following IP injection of PBS, and solid tumors excised from the omental region of animals. RBC lysis was performed on blood and peritoneal wash samples using ACK lysis buffer and solid tumors were processed using the gentleMACs Dissociator (Miltenyi Biotec), followed by passage through 70 m cell strainers. Samples were subsequently stained for flow cytometry analysis as outlined.

### Metabolomic Assessment of FR-B T Cells Using Seahorse

FR-B 2/7 or FR-B 2/15 T cells were generated for comparison of metabolic function using the Mitochondrial Stress Test conducted using the Seahorse XFe96 Analyzer. Briefly, the Mitochondrial Stress Test was performed in XF DMEM Base Media with no Phenol red containing 10 mM glucose, 1 mM sodium pyruvate, and 2 mM L-glutamine and the following inhibitors were added at the following final concentrations: Oligomycin (2 μM), Carbonyl cyanide 4-(trifluoromethoxy)phenylhydrazone (FCCP) (1.0 μM), Rotenone/Antimycin A (0.5 μM each). Prepared T cells were plated on cell-tak (Corning) coated Seahorse XF96 cell culture microplates at a density of 2.5×10⁵ cells per well and in replicates of 8 wells per cell type. The assay plates were allowed to rest at RT for 45 mins and then spun for 5 minutes at 1,000 rpm. The plates were then incubated at 37° C. without CO2 for 10 mins prior to performing the assay on the Seahorse XFe96 Analyzer. Assay was set up and ran according to manufacturer's recommended method and post run data was obtained through Seahorse Wave Desktop Software, with additional statistical analysis completed using GraphPad Prism.

### FACs Sorting of CD8+FR-B TALs, RNA Sequencing, and Transcriptomic Analysis

FR-B 2/7 or FR-B 2/15 T cells (8.4×10⁵/mouse, prepared in 200 μl PBS) were adoptively transferred by IP injection into IE9-mp1-hFRa tumor-bearing mice and peritoneal washes collected from mice 5 days later following IP injection of 5 ml PBS. Collected cells were washed and immediately stained for cell viability (Zombie UV, prepared in PBS) followed by Fc blocking and surface phenotyping using antibodies prepared in BD FACs Pre-Sort Buffer (BD Biosciences). Live CD45+CD11b⁻CD19⁻CD4⁻ (Gated out using a PE-Cy7 Dump Channel) CD8+ GFP+FR-B TALs were sorted using a BD FACSAria II Cell Sorter and collected directly into PCR tubes containing 2 μl of Takara Plain Sorting Solution with a target collection of 500 cells/sample. Sufficient cell input (359-500 cells) was achieved for downstream analysis using 5 unique biological samples (n=2 for FR-B 2/7 CD8+ TALs, n=3 for FR-B 2/15 CD8+ TALs). Sorted cells were kept on ice and immediately brought to the Roswell Park Genomics Shared Resource for further processing and RNAseq analysis. RNAseq analysis of sorted CD8+FR-B TALs was conducted using the Takara Bio USA, Inc. SMART-Seq® v4 PLUS Kit. Final libraries were sequenced on an Illumina NovaSeq 6000 using 2×100 sequencing and an average of 50 million paired reads/sample were generated. Following sequencing, samples were passed through Illumina bcl2fastq v2.20 to generate fastq files for downstream analysis. Bioinformatics pre-processing and quality control (QC) steps were carried out by the Roswell Park Bioinformatics Shared Resource, using an established pipeline following commonly adopted practices for RNA-seq data analysis. Raw reads that passed the Illumina RTA quality filter were demultiplexed and pre-processed using FastQC for sequencing base quality control. Raw reads that passed the Illumina RTA quality filter were demultiplexed and pre-processed using FastQC for sequencing base quality control. Reads were then mapped to the mouse reference genome (GRCm39) and reference transcriptome GENCODE (vM28) using STAR⁶. Raw feature counts were normalized and differential expression analysis was carried out using DESeq2⁷. Differential expression rank order was used for subsequent gene set enrichment analysis (GSEA)⁸, performed using the cluster profile package in R. Gene sets queried included the Hallmark, Canonical pathways, and GO Biological Processes Ontology collections available through the Molecular Signatures Database (MSigDB)⁹.

### SUPPLEMENTAL REFERENCES

1. Tsuji T, Yoneda A, Matsuzaki J, et al. Rapid Construction of Antitumor T-cell Receptor Vectors from Frozen Tumors for Engineered T-cell Therapy. Cancer Immunol Res 2018; 6(5):594-604. doi: 10.1158/2326-6066.Cir-17-0434 [published Online First: 2018/03/29]
2. Gilliland L K, Norris N A, Marquardt H, et al. Rapid and reliable cloning of antibody variable regions and generation of recombinant single chain antibody fragments. Tissue Antigens 1996; 47(1):1-20. doi: 10.1111/j.1399-0039.1996.tb02509.x [published Online First: 1996/01/01]
3. Chewning J H, Dugger K J, Chaudhuri T R, et al. Bioluminescence-based visualization of CD4 T cell dynamics using a T lineage-specific luciferase transgenic model. BMC Immunol 2009; 10:44. doi: 10.1186/1471-2172-10-44 [published Online First: 2009/08/05]
4. McGray A J R, Eppolito C, Miliotto A, et al. A prime/boost vaccine platform efficiently identifies CD27 agonism and depletion of myeloid-derived suppressor cells as therapies that rationally combine with checkpoint blockade in ovarian cancer. Cancer Immunol Immunother 2021; 70(12):3451-60. doi: 10.1007/s00262-021-02936-1 [published Online First: 2021/04/22]
5. McGray A J R, Huang R Y, Battaglia S, et al. Oncolytic Maraba virus armed with tumor antigen boosts vaccine priming and reveals diverse therapeutic response patterns when combined with checkpoint blockade in ovarian cancer. J Immunother Cancer 2019; 7(1):189. doi: 10.1186/s40425-019-0641-x [published Online First: 2019/07/19]
6. Dobin A, Davis C A, Schlesinger F, et al. STAR: ultrafast universal RNA-seq aligner. Bioinformatics 2013; 29(1):15-21. doi: 10.1093/bioinformatics/bts635 [published Online First: 2012/10/30]
7. Love M I, Huber W, Anders S. Moderated estimation of fold change and dispersion for RNA-seq data with DESeq2. Genome Biol 2014; 15(12):550. doi: 10.1186/s13059-014-0550-8 [published Online First: 2014/12/18]
8. Reimand J, Isserlin R, Voisin V, et al. Pathway enrichment analysis and visualization of omics data using g:Profiler, GSEA, Cytoscape and EnrichmentMap. Nat Protoc 2019; 14(2):482-517. doi: 10.1038/s41596-018-0103-9 [published Online First: 2019/01/22]
9. Liberzon A, Subramanian A, Pinchback R, et al. Molecular signatures database (MSigDB) 3.0. Bioinformatics 2011; 27(12):1739-40. doi: 10.1093/bioinformatics/btr260 [published Online First: 2011/05/07]

1. A modified cell that is modified to express and secrete a Bi-specific T cell engager (BiTE), wherein the BiTE includes segment that specifically binds to human Folate Receptor alpha (FRα) and a segment that that specifically binds to human CD3, wherein the human CD3 is optionally CD3e.

2. The modified cell of claim 1, wherein the modified cell is a modified human T cell.

3. The modified human T cell of claim 2, wherein the segment that specifically binds to the human Folate Receptor alpha (FRα) comprises amino SEQ ID NO:6, and wherein the segment that specifically binds to human CD3 specifically binds to human CD3e and comprises the sequence of SEQ ID NO:5.

4. The modified human T cell of claim 2, wherein the BiTE comprises amino acids 1-531 of SEQ ID NO:10 and wherein the BiTE is secreted from the modified human T cell.

5. A method comprising introducing into an individual in need of treatment for cancer modified human cells of claim 1.

6. The method of claim 5, wherein the modified human cells are T cells.

7. The method of claim 6, wherein in the modified human T cells the segment of the Bi-specific T cell engager (BiTE) that specifically binds to the human Folate Receptor alpha (FRα) comprises amino SEQ ID NO:6, and wherein the segment that specifically binds to human CD3 specifically binds to human CD3e and comprises the sequence of SEQ ID NO:5.

8. The method of claim 7, wherein the BiTE comprises amino acids 1-531 of SEQ ID NO:10.

9. The method of claim 8, wherein the cancer comprises a solid tumor.

10. The method of claim 9, wherein the solid tumor comprises ovarian cancer cells.

11. The method of claim 10, wherein the modified human T cells are administered a single time and have therapeutic efficacy.

12. The method of claim 10, wherein the modified human T cells are administered in consecutive doses, and wherein the consecutive doses are optionally separated by a period of time comprising more than 24 hours, and wherein said doses have therapeutic efficiency.

13. A method of making modified human cells that express and secrete a Bi-specific T cell engager (BiTE) as in claim 1, the method comprising conditioning the cells in a medium that comprises interleukin (IL) IL-2, IL-7, IL-15, or a combination thereof, and introducing into the cells a polynucleotide encoding the BiTE, wherein the conditioning is performed prior to introduction of the polynucleotide, concurrent with introduction of the polynucleotide, or after introduction of the polynucleotide.

14. The method of claim 13, wherein the modified human cells are T cells.

15. Modified human cells made according to the method of claim 13.

16. A method comprising administering to an individual in need of treatment for cancer modified human cells of claim 13.

17. The method of claim 16, wherein the individual is in need of treatment for ovarian cancer.

18. A polynucleotide that encodes a Bi-specific T cell engager (BiTE) as in claim 1.

19. The polynucleotide of claim 18, wherein the polynucleotide is present in a viral expression vector.

20. The polynucleotide of claim 19, wherein the viral vector is a retroviral vector.
